(19) United States
(12) Patent Application Publication
(10) Pub. No.: US 2023/0050868 A1
(43) Pub. Date: Feb. 16, 2023

(54) METHODS AND COMPOSITIONS FOR PREVENTING ATOPIC MARCH AND TREATING SKIN CONDITIONS

(71) Applicant: Yobee Care, Inc., Chicago, IL (US)

(72) Inventor: Tarun Jain, Chicago, IL (US)

(21) Appl. No.: 17/938,393

(22) Filed: Oct. 6, 2022

Related U.S. Application Data
(63) Continuation of application No. PCT/US2021/028226, filed on Apr. 20, 2021.
(60) Provisional application No. 63/013,684, filed on Apr. 22, 2020.

(51) Int. Cl. A61K 35/747; A61K 35/644; A61K 36/9066; A61K 47/55

(52) U.S. Cl. CPC A61K 35/747; A61K 35/644; A61K 36/9066; A61K 47/551

(57) ABSTRACT

Provided herein are methods and compositions for preventing atopic march and treating a skin condition.

### BACKGROUND

The present application claims priority to PCT application serial no. PCT/US2021/028226, filed Apr. 20, 2021, which claims priority to U.S. provisional application Ser. No. 63/013,684, filed Apr. 22, 2020, each herein incorporated by reference in their entireties.

### BACKGROUND

The invention generally relates to prevention and treatment of allergic diseases.

The natural history of atopic manifestations is referred to as “Allergic March” or “Atopic March” and is characterized by a typical sequence of immunoglobulin E (IgE) antibody responses and clinical symptoms which may appear early in life, persist over years or decades and often remit spontaneously with age. The allergic diseases that often begin early in life include atopic dermatitis (AD), food allergy, asthma and allergic rhinitis. In general, no clinical symptoms except a dry skin are detectable at birth. The production of IgE starts in the 11th week of gestation, no specific sensitization to food or inhalant allergens can be detected in cord blood with standard methods for measuring elevated serum IgE antibodies. The first IgE responses directed to food proteins may be observed during the first weeks of life. First IgE responses are most commonly directed to proteins from hen's egg, peanuts and cow's milk, independent of the mode of feeding (breastfeeding versus formula feeding). These strong infantile IgE antibody responses to food proteins can be considered as markers for atopic reactivity, since they have been demonstrated to be predictors of subsequent sensitization to other food proteins (peanuts, tree nuts) or aeroallergens from the indoor or outdoor surroundings. Sensitization to environmental allergens and other food allergens like tree nuts and shellfish require additional time and are observed during the pre-school or early school-age period. In numerous atopic individuals, atopic dermatitis (AD) is the first clinical manifestation with the highest incidence during the first months of life, and the highest period occurrence during the first three years of life. Over 50% of patients with AD develop respiratory allergies such as asthma and allergic rhinitis.

AD is a chronic inflammatory skin disorder that affects nearly 17% of children and can continue into adulthood. Understanding the mechanisms underlying AD require direct sampling of AD skin. Although AD is primarily a skin disease involving infants and young children, there are little skin-based studies examining AD in this age group because of the invasiveness of skin biopsies. AD is often associated with food allergy and asthma. The abnormal skin barrier in patients with AD may allow epicutaneous absorption of environmental allergens through the skin and promote systemic allergen sensitization, which predisposes to the development of food allergy and asthma (Broccardo C J, et al. Peeling off the layers: skin taping and a novel proteomics approach to study atopic dermatitis. J Allergy Clin Immunol 2009; 124:1113-5 e 1-11; Leung D Y, Guttman-Yassky E. Deciphering the complexities of atopic dermatitis: shifting paradigms in treatment approaches. J Allergy Clin Immunol 2014; 134:769-79).

Because current treatment approaches are not curative, there is considerable interest in studying approaches to prevent AD as well as other infantile allergic diseases, including use of strategies to improve skin barrier or downregulate the type 2 allergic immune response. This may be due to a lack of standardization of the bacterial preparations or lack of biomarkers to identify which AD phenotype would benefit from this approach (Broccardo C J, et al. Peeling off the layers: skin taping and a novel proteomics approach to study atopic dermatitis. J Allergy Clin Immunol 2009; 124:1113-5 el-11; Leung D Y, Guttman-Yassky E. Deciphering the complexities of atopic dermatitis: shifting paradigms in treatment approaches. J Allergy Clin Immunol 2014; 134:769-79).

The present invention attempts to solve this problem, as well as others.

### SUMMARY OF THE INVENTION

Provided herein are methods and compositions for preventing atopic march.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

As used herein, the term “subject” may be used interchangeable with the terms “patient” and “individual.” A subject may include a human and/or a non-human animal (e.g., a companion animal such as a dog or a cat).

Generally speaking, the method comprises providing bacteria and honey to the skin, scalp, or hair of a subject to provide a barrier layer in normal healthy skin, scalp, or hair; and preventing eczema and atopic march development in the subject.

There is an increasing body of evidence supporting the role of an inflamed, disrupted skin barrier being the route of allergic sensitization to foods and potentially also respiratory allergens.^{1,2}. There is strong evidence that FLG mutations, which have an important role in formation and integrity of the outer layer of the skin (stratum corneum) are associated with eczema.³ Filaggrin null mutations also increase the risk of eczema, FA, and asthma.^{4,5} Furthermore, Type 2 skin inflammation which occurs in eczema, can lead to secondary reduction in the protein filaggrin in the skin, leading to a vicious cycle of inflammation and skin barrier disruption.⁶ Mouse studies demonstrate that exposure to food allergens through abraded skin, mimicking eczema, induces allergic sensitization.⁷ In vitro evidence has shown that FA correlates with specific proliferation in the skin-homing memory T cell subset, suggesting that sensitization is occurring though the skin.^{8,9} High levels of environmental food exposure are associated with increased sensitization and allergy in children with an impaired skin barrier^{10,11}

There are limitations of oral tolerance induction in the prevention of food allergy. Early oral peanut ingestion from 4-11 months of age reduced the risk of school-age FA, still the children became allergic to tree nuts and sesame seed even with oral ingestion.¹² Food allergy develops early in life; thus there appears to be a narrow window of opportunity to prevent the development of FA and little time to include multiple foods into infants' diet; for example a high proportion of egg allergy has already developed by 6 mo.^{13,14}

The mechanistic role of IL-33 and TSLP have been studied in several animal models of atopic diseases. Stimuli such as allergens, bacteria, mechanical injury, and environmental factors trigger the release of IL-33 and TSLP from various cell types. IL-33 and TSLP, alone or in combination with other cytokines and chemokines, subsequently activates Th2 cells, Type 2 innate lymphoid cells (ILC), mast cells, dendritic cells, basophils, eosinophils and macrophages triggering the release of further cytokines, chemokines, and other pro-inflammatory mediators involved in the pathophysiology of atopic and other inflammatory diseases, and leading to IgE class switching by B cells.

With growing evidence that food sensitization can occur through the skin, more attention has also been paid to dysbiosis of the skin microbiome as a factor both in eczema and FA. Skin commensals have been found to be essential for resident T cells and educate keratinocytes to become effective in combating skin pathogens. Conversely, commensals also appear to be required so that the host does not attack normal colonizing flora. Dysbiosis of the skin microbiome has been observed in infants with eczema,^{15,16} with staphylococcal species, particularly S. aureus, appearing to play an important role in this process.¹⁷

In examining local tolerance mediated by regulatory cells (CD8+ Tcells, Treg, Breg), many lines of evidence suggest that these are antigen specific regulatory cells35-38 and that they have specific homing receptors to the gut and skin.^{18,19-21} In addition, mediators such as IL-10, PPAR-γ, PPAR-α, and amphiregulin have been shown to be important to mediate skin barrier protection and homeostasis with the immune system.^{22,23} The effects of these cell populations on the numbers of IgE+B lineage cells, and the production and avidity of allergen-specific IgE antibodies, or the induction of potentially protective antibody isotypes such as allergen-specific IgG4, remain to be studied.

In one embodiment, the method comprising providing bacteria and honey to the skin, scalp, or hair of a subject with an skin condition selected from eczema, cradle cap, and scalp conditions; providing a barrier layer to food antigens and/or environmental antigens from entering thru skin in an infant; and preventing food allergies, asthma, or environmental allergies.

In another embodiment, the method comprises providing bacteria and honey to the skin, scalp, or hair of a subject in combination with topical steroids, oral steroids, and/or EpiCeram to provide a synergistic treatment for preventing eczema, atopic march, allergies, asthma, or environmental allergies.

In another embodiment, the method comprises providing bacteria and honey to the skin, scalp, or hair in combination with a food allergy primary prevention therapy including the introduction of allergenic foods selected from the group consisting of: peanuts, tree nuts, shellfish, finfish, milk, eggs, soy, wheat, sesame, legumes, corn, fruits, vegetables, meats, seeds; and providing a synergistic treatment for preventing eczema, atopic march, allergies, asthma, or environmental allergies.

In one embodiment, the method comprises providing honey and bacteria to the skin, scalp, or hair; and providing a food allergy secondary prevention therapy selected from the group consisting of: oral immunotherapy, epicutaneous immunotherapy, and sublingual immunotherapy; and providing a synergistic treatment preventing eczema, atopic march, allergies, asthma, or environmental allergies. The oral immunotherapy may be Palforzia, according to one embodiment. The epicutaneous immunotherapy may be Viaskin's skin patches including a dry layer of allergen in its center and the patch is positioned on intact skin, according to one embodiment. The sublingual immunotherapy may be Allergy Tablets used under the tongue to boost tolerance to the allergy to and reduce symptoms, according to one embodiment.

In another embodiment, the method comprises providing honey and bacteria to the skin, scalp, or hair in combination with prebiotics or probiotics; and providing a synergistic treatment for preventing eczema, atopic march, allergies, asthma, or environmental allergies. Prebiotics are compounds in food that induce the growth or activity of beneficial microorganisms such as bacteria and fungi. The most common example is a type of fiber that the human body cannot digest. Probiotics, which are tiny living microorganisms, including bacteria and yeast, where prebiotics can alter the composition of organisms in the gut microbiome. Additional probiotics are listed below.

In another embodiment, the method comprises providing honey and bacteria to the skin, scalp, or hair in combination with a food allergy vaccine; and providing a synergistic treatment for preventing eczema, atopic march, allergies, asthma, or environmental allergies. Food allergy vaccine may be an allergen immunotherapy, also known as allergy shots, is a form of long-term treatment that decreases symptoms for many people with allergic rhinitis, allergic asthma, conjunctivitis (eye allergy) or stinging insect allergy. Allergy shots decrease sensitivity to allergens and often leads to lasting relief of allergy symptoms even after treatment is stopped.

In another embodiment, the method comprises providing honey and bacteria to the skin, scalp, or hair in combination with biologics/immunomodulators; and providing a synergistic treatment for preventing eczema, atopic march, allergies, asthma, or environmental allergies. In one embodiment, the biologic s/immunomodulators are selected from the group consisting of: Omalizumab and/or Dupilumab.

In another embodiment, the method comprises providing honey and bacteria to the skin, scalp, or hair in combination with other skin condition treatments; and providing a synergistic treatment for preventing eczema, atopic march, allergies, asthma, or environmental allergies. The skin condition treatments treat or cure skin conditions selected from the group consisting of: dry skin, atopic dermatitis, eczema, psoriasis, cradle cap, Hair loss, Allergic reactions, contact dermatitis, Acne, Rosacea, Oily skin, stress-related skin conditions, Progressive macular hypomelanosis, Skin infection, fungal infections, athletes foot, sores, Chronic blistering, burns, insect bites, scars, Conditions of the mucous membranes, Conditions of the skin appendages, Erythemas, necrotizing cellulitis, cutaneous anthrax, cellular weaving, erysipelas, smallpox, necrotizing fasciitis, gangrene, sepsis, pyoderma, endocarditis, Skin transplants, and skin grafts.

The method comprises preventing or delaying the development of atopy or the atopic march in a subject by applying bacteria to the skin, scalp, and/or hair of the subject. In one embodiment, the method comprises preventing or reducing severity or incidence of allergic immune response(s) to an allergen in a mammalian subject by applying bacteria and honey to the skin, scalp, and/or hair of the subject, or preventing or attenuating severity of allergic disease, such as airway hyper-responsiveness in a mammalian subject following exposure of the subject to an allergen by applying bacteria to the skin, scalp, and/or hair of the subject. The method comprises preventing or interrupting or limiting the atopic march and progression of an allergic disease such as eczema in children e.g., neonates and juveniles to food allergy and/or severe asthma later in life for example during adolescence and/or adulthood, by applying bacteria to the skin, scalp, and/or hair.

Bacteria may be selected from the group consisting of Lactobacillus, Bifzdobacterium, or Streptococcus. In one embodiment, the bacteria may belong to a species selected from Lactobacillus acidophilus, Lactobacillus plantarum, Lactobacillus rhamnosus, Lactobacillus delbruecki, Lactobacillus paracasei, Lactobacillus salivarius, Lactobacillus casei, Bifidobacterium lactis, Bifidobacterium longum, Bifidobacterium breve, and Streptococcus thermophiles.

Administration of a composition in the early post-natal period comprises bacteria to the skin, scalp, and/or hair prevents the atopic march in a subject and development of allergy in adolescents and adults is optimally deliverable to neonates or during early childhood. Administration of the composition in all procedures, steps, examples, and methods described herein may be combined with any of the following methods, in any order, or combination thereof:
1. Providing a barrier layer to food antigens and/or environmental antigens from entering thru skin in an infant to prevent food allergies, asthma, or environmental allergies;
2. Providing the composition in combination with topical steroids, oral steroids, and/or EpiCeram to provide a synergistic treatment for preventing eczema, atopic march, allergies, asthma, or environmental allergies;
3. Providing the composition in combination with a food allergy primary prevention therapy to provide a synergistic treatment for preventing eczema, atopic march, allergies, asthma, or environmental allergies;
4. Providing the composition with a food allergy secondary prevention therapy selected from the group consisting of: oral immunotherapy, epicutaneous immunotherapy, and sublingual immunotherapy to provide a synergistic treatment for preventing eczema, atopic march, allergies, asthma, or environmental allergies;
5. Providing the composition in combination with prebiotics or probiotics to provide a synergistic treatment for preventing eczema, atopic march, allergies, asthma, or environmental allergies;
6. Providing the composition in combination with a food allergy vaccine to provide a synergistic treatment for preventing eczema, atopic march, allergies, asthma, or environmental allergies;
7. Providing the composition in combination with biologics/immunomodulators to provide a synergistic treatment for preventing eczema, atopic march, allergies, asthma, or environmental allergies; or
8. Providing the composition in combination with a skin treatment to provide a synergistic treatment for preventing eczema, atopic march, allergies, asthma, or environmental allergies.

In one embodiment, the composition comprises inactivated and/or killed bacteria administered to the skin, scalp, or hair and of neonates or adults of allergy to reduce the incidence or severity of an allergic response to antigenic challenge. An antigenic challenge may be a measurement of airway or lung resistance. In one embodiment, administration of an inactivated and/or killed bacteria to the skin, scalp, or hair, wherein the inactivated bacteria does not have the same capacity of a live bacteria to colonize the skin, scalp, or hair of a mammal to which it is administered or wherein the inactivated or killed bacteria is incapable of colonizing the skin, scalp, or hair of a mammal to which it is administered, or a bacteria cell lysate, appears to interrupt or slow or arrest or prevent atopic march or further atopic march in the subject e.g., by delaying or preventing or interrupting or slowing the onset of one or more allergic conditions such as allergic eczema, urticaria, hives, rhinitis, wheezing, airway resistance, airway restriction, or airway hyper-responsiveness or hyper-reactivity, food allergy, asthma, and the like.

For one embodiment, by administering inactivated and/or killed bacteria to the skin, scalp, or hair e.g., isolated inactivated and/or killed bacteria, to a subject that is asymptomatic for eczema, or asymptomatic for allergy e.g., characterized by rhinitis or wheezing or airway resistance or restriction or airway hyper-responsiveness, or asymptomatic for asthma, a subsequent onset of eczema and/or allergy and/or asthma may be prevented. In one embodiment, inactivated and/or killed bacteria is administered to the skin, scalp, or hair of a juvenile subject such as a neonate or infant to prevent eczema in the infant or a subsequent onset of allergy or asthma in later life e.g., in adolescence or adulthood. In another embodiment, inactivated and/or or killed bacteria e.g., isolated inactivated and/or killed bacteria, is administered to the skin, scalp, or hair of an adolescent or adult subject to prevent eczema in the subject or a subsequent onset of allergy or asthma, such as in later life. This is in a background in which allergic eczema, allergy or asthma is inducible at any stage of life by exposure of a subject to one or more challenge allergens, including one or more environmental allergens e.g., pollen allergen, dust mite allergen, animal allergen, chemical allergen, and the like.

Alternatively, by administering inactivated and/or killed bacteria e.g., isolated inactivated and/or killed bacteria, to the skin, scalp, or hair of a subject that has suffered previously from one or more incidences of allergic eczema, allergy e.g., characterized by rhinitis or wheezing or airway resistance or restriction, or asthma, a subsequent attack may be prevented or the severity of a subsequent attack may be reduced. In one embodiment, inactivated and/or killed bacteria e.g., isolated inactivated and/or killed bacteria is administered to a juvenile subject that has suffered from allergic eczema to prevent a subsequent attack or reduce severity of a subsequent attack, optionally to prevent or slow further atopic march in the subject. In another embodiment, inactivated and/or killed bacteria e.g., isolated inactivated and/or killed H. bacteria is administered to the skin, scalp, or hair of an adolescent or adult subject that has suffered previously from allergic eczema and/or allergy and/or asthma, to prevent a subsequent attack or reduce severity of a subsequent attack, optionally to prevent or slow further atopic march in the subject.

In an epidemiological context, the administration of inactivated and/or killed bacteria e.g., isolated inactivated and/or killed bacteria to the skin, scalp, or hair of a subject reduces the incidence of allergic immune responses in the population, and reduces the incidence of allergic immune responses in adolescent and/or adult members of the population treated when they were juveniles.

Inactivated and/or killed bacteria protect subjects by avoiding health risks associated with the use of live bacteria. In other words, inactivated and/or killed bacteria or a lysate of bacteria offers a safe and controlled approach for positively-influencing the developing immune system, and preventing or reducing an allergic response to an allergen. Similarly, inactivated and/or killed bacteria or a lysate of bacteria offers a safe and controlled approach to delaying or preventing the atopic march by targeting events in early in life e.g., in children such as neonates and/or juveniles.

The administration or repeated administration, of inactivated and/or killed bacteria and/or a bacteria lysate thereof e.g., to children or infants such as at about 0 to about 5 years of age, to thereby promote balanced immune development for reducing the severity or incidence of allergy e.g., as allergic eczema and/or a life-long food allergy and/or allergic asthma. The inactivated and/or bacteria and/or a lysate thereof is also useful for modulating the immune system of a mammalian subject and/or for improving the immune system's tolerance to allergy.

As disclosed herein, the inactivated and/or killed bacteria and/or a lysate thereof are formulated as a topical composition, as described below. Such topical compositions for improving immune system's tolerance to allergens and/or preventing or reducing allergy symptoms for example in adults and/or adolescents. The compositions are preferably for repeated administration, e.g., daily, to children and/or infants, e.g., aged about 0 to about 5 years, suffering from eczema and/or food allergy or susceptible to development of eczema or food allergy. In this respect, a subject may be susceptible to development of allergy at about 0 to about 5 years or about 0 to about 4 years or about 0 to about 3 year or about 0 to about 2 years or about 0.1 to about 5 years or about 0.1 to about 4 years or about 0.1 to about 3 years or 0.1 to about 2 years or about 0.1 to about 1 years or about 1 to about 2 years or about 1 to about 3 years or about 1 to about 4 years or about 1 to about 5 years or about 2 to about 3 years or about 2 to about 4 years or about 2 to about 5 years or about 3 to about 4 years or about 3 to about 5 years of age. For one embodiment, to prevent or limit the atopic march in a subject, such as progression to food allergy and/or allergic asthma later in life, the subject is administered a plurality of doses of a composition comprising the inactivated bacteria or cell extract or lysate thereof, wherein the first does is administered at a time infra where the subject is susceptible to development of allergy. For one embodiment, the subject may be taking antibiotic therapy or prescribed antibiotic therapy, especially in the case of an infant or child that is susceptible to development of allergy.

Mechanism of Action

Without being bound by theory or specific mode of action, the inactivated and/or killed bacteria facilitate immune modulation in a subject towards a balanced immune response to an allergen e.g., balanced Th1/Th2 immune response in a subject and/or to interrupt or slow or arrest or prevent atopic march or further atopic march in the subject e.g., by delaying or preventing or interrupting or slowing the onset of one or more allergic conditions described herein.

The healthy skin has an intact barrier and a balanced microbiome. Subjects with skin conditions can have a damaged barrier and/or an unbalanced microbiome. The honey creates a protective skin barrier that prevents allergen entry thru the damaged skin that may otherwise lead to an abnormal immune response that may lead to various atopic conditions. The anti-inflammatory properties of honey may also reduce an abnormal immune response within the eczema skin lesions. Turmeric has anti-inflammatory properties and may also reduce an abnormal immune response within the eczema skin lesions. Lipids and or ceramides may have similar function of creating a protective skin barrier against allergen entry thru the damaged skin. Diseased skin, including eczematous skin, may have lower ceramide levels in the stratum corneum. The use of prebiotics and/or probiotics may enhance the growth of healthy bacteria on the skin, which may provide protection against allergen entry thru the damaged skin.

A healthy skin microbiome prefers a relatively acidic environment (pH 4.5-5.5) which may also inhibit growth of pathogens. Skin pH that is too elevated can contribute to impaired skin barrier function. This may be seen in patients with eczema, where an elevated pH contributes to impaired barrier dysfunction and an environment favoring the growth of bacteria such as S. pyogenes and S. aureus, contributing to dysbiosis of the skin's microbiome. Inactivated and/or killed bacteria may help rebalance skin microbiome and thus normalize the skin pH. Oral and/or topical prebiotic and/or probiotic use may also normalize the skin pH.

Composition

Topical compositions with the bacteria are formulated in a suitable base vehicle comprising honey for applying bacteria to the skin, scalp, and hair as a topical composition. The disclosed topical compositions preferably are formulated to be supportive and compatible with the skin microbiome and pH.

In particular, the base vehicle may include honey as produced by honey bees including, but not limited to, honey produced by any species of the genus Apis such as A. mellifera, A. cerana, A. florea, A. andreniformis, A koschevnikovi, and A. dorsata. The base vehicle may include honey produced by honey bees and collected pollen and nectar from any flowering plant, in particular, honey produced from the pollen and nectar of Leptospermum scoparium and its relatives and/or the Manuka tree or its relatives (i.e., Mnuka honey). Suitable honey for the disclosed topical compositions may include raw honey or processed honey. In some embodiments, honey used in the disclosed compositions has been heat-treated (e.g., via pasteurization) and/or irradiated.

The disclosed topical compositions include a suitable concentration of honey, for example, to prepare a composition for treating and/or preventing skin conditions and/or scalp conditions. In some embodiments, the honey may be present in the disclosed compositions at a concentration of at least about 1%, 2%, 3%, 5%, 7%, 10%, 15%, 20%, 25%, 30%, 35%, 40%, 45%, 50%, 55%, 60%, 65%, 70% (w/w) or higher, or within a range bounded by any of these values (e.g. about 15-60% (w/w) or about 20-50% (w/w)).

The disclosed topical compositions include bacteria which may be live bacteria and/or killed bacteria and/or inactivated bacteria. Killed bacteria for the disclosed compositions may be killed by methods known in the art, including but not limited to heat-treatment, irradiations, and/or chemical treatment. In some embodiments, the bacteria are present in the topical composition at a concentration of at least about 10⁴, 10⁵, 10⁶, 10⁷, 10⁸ colony forming units (CFU)/gram (g) topical composition or higher, or within a concentration range bounded by any of these values (e.g., 10⁴-10⁸ CFU/g). Where the bacteria are killed bacteria, the concentration of the bacteria added to the composition may be determined prior to killing the bacteria. In other embodiments, the bacteria are present at a concentration of at least about 10⁻⁸, 10⁻⁷, 10⁻⁶, 10⁻⁵, 10⁻⁴ g bacteria/g composition or higher, or within a concentration range bounded by any of these values (e.g., 10⁻⁷-10⁻⁸ g bacteria/g composition). In other embodiments, the bacteria are present at a concentration of at least about 1%, 2%, 3%, 5%, 7%, 10%, 15%, 20% (w/w) or higher or within a range bounded by any of these values (e.g., at a concentration of 3-10% (w/w)).

In some embodiments, bacteria for the disclosed topical compositions may include bacteria suitable for use in a topical formulation for treating and/or preventing skin conditions and/or scalp conditions. Suitable bacteria for the disclosed compositions may include, but are not limited to bacteria of the genera Lactobacillus, Bifidobacterium, or Streptococcus, especially Lactobacillus acidophilus, Lactobacillus plantarum, Lactobacillus rhamnosus, Lactobacillus delbruecki, Lactobacillus paracasei, Lactobacillus salivarius, Lactobacillus casei, Bifidobacterium lactis, Bifidobacterium longum, Bifidobacterium breve, and Streptococcus thermophiles.

Optionally, the disclosed topical compositions may include additional bacterial species or non-bacterial species that natural occur in honey (e.g. yeasts), products of fermentation (e.g. lactic acid and/or ethanol), peroxides and any other metabolic byproducts of the bacteria listed above, or of those organisms contained in honey, and additional carriers, buffers, emulsifiers, and anti-oxidants. Alternatively, the honey may be pasteurized or be a spore-free and/or toxin-free honey (such as spores or toxins of Clostridium Botulinum).

Additional components for the disclosed topical compositions may include, but are not limited to plant-based products. In some embodiments, the one or more plant-based products are present in the topical composition at a concentration of at least about 5%, 10%, 20%, 30%, 40%, 50%, 55%, 60%, 65%, 70%, 75%, 80%, 85%, 90% or higher, or within a concentration range bounded by any of these values (e.g., 30-90% (w/w), or about 30-70% (w/w), or about 30-50% (w/w)).

Suitable plant-based products for the disclosed topical compositions may include plant-based lipid products. Plant-based lipid products may include plant-based butters (such as shea butter or cocoa butter), plant-based waxes (such as carnauba wax or beeswax), and plant-based oils (such as coconut oil, sunflower oil, or jojoba oil, or fractions thereof).

Suitable plant-based products for the disclosed topical compositions may include plant-based gels, lotions, or other extracts or components. In some embodiments, the disclosed composition comprises a plant-based gel, lotion, or other extract or component from the Aloe vera plant or plants producing similar substances as the Aloe vera plant.

Additional plant-based components for the disclosed topical compositions may include turmeric (e.g., Curcuma longa), turmeric-derived products, or components that are present in turmeric. In some embodiments, the disclosed compositions include turmeric powder and/or components that are present in turmeric such as curcuminoids and essential oils. The disclosed compositions may include components selected from but not limited to curcumin, demethoxycurcumin, and bisdemethoxycurcumin. The disclosed compositions may include components selected from but not limited to turmerone, germacrone, atlantone, and zingiberene. In some embodiments, the disclosed compositions comprise turmeric-derived products, or components that are present in turmeric at a concentration of at least about 0.5%, 1.0%, 1.5%, 2.0%, 2.5%, 3.0%, 3.5%, 4.0%, 4.5%, 5.0%, 5.5%, 6.0%, 6.5%, 7.0%, 7.5%, 8.0%, 8.5%, 9.0%, 9.5%, 10.0% or higher, or within a concentration range bounded by any of these values (e.g., about 0.5-10% (w/w) or about 2.0-6.0% (w/w)).

The disclosed topical compositions may comprise curcuminoids or essential oils (e.g., curcuminoids and essential oils that are present in turmeric (such as curcumin, demethoxycurcumin, and bisdemethoxycurcumin; and turmerone, germacrone, atlantone, and zingiberene)). In some embodiments, the curcuminoids and/or essential oils are present in the composition at a concentration of at least about 0.005%, 0.01%, 0.02%, 0.05%, 0.1%, 0.2%, 0.5% or higher, or within a concentration range bounded by any of these values (e.g., about 0.005-0.5% (w/w) or 0.05-0.5% (w/w)).

Additional plant-based components for the disclosed topical compositions may include, but are not limited to beta-glucans, olive polyphenols, ahi flower oil, carotenoids such as fucoxanthin, ceramides, and fatty acids such as omega-7 oil optionally obtained from sea buckthorn.

The disclosed topical compositions may comprise micronutrient components, including but limited to vitamins. Suitable vitamins may include, but are not limited to vitamin B12, vitamin E and/or vitamin B3. In some embodiments, the micronutrient (e.g., vitamin B12, vitamin E and/or vitamin B3) is present in the topical composition at a concentration of at least about 0.1%, 0.2%, 0.5%, 1.0%, 1.5%, 2.0%, 2.5%, 3.0%, 3.5%, 4.0%, 4.5%, 5.0%, 5.5%, 6.0% or higher, or within a concentration range bounded by any of these values (e.g., about 0.1-5% (w/w) or about 1-3% (w/w)).

The disclosed topical compositions may comprise a humectant. Suitable humectants may include, but are not limited to glycerin, or other fractions of triglyceride hydrolysis. In some embodiments, the disclosed compositions comprise glycerin (or other fractions of triglyceride hydrolysis) at a concentration of about 1%, 2%, 3%, 5%, 10%, 15%, 20%, 25%, 30% or higher, or within a concentration range bounded by any of these values (e.g., 3-30% (w/w), or about 5-25% (w/w), or about 10-20% (w/w), or about 15% (w/w)).

The disclosed topical compositions may comprise emulsifiers. Suitable emulsifiers may include, but are not limited to, plant-based emulsifiers such as lecithin, especially from Helianthus annuus.

The disclosed topical compositions may include anti-oxidants. Suitable anti-oxidants may include, but are not limited to, hydroxytyrosol.

Additional components for the disclosed topical compositions may include, but are not limited to beta-glucans, olive polyphenols, ahi flower oil, carotenoids such as fucoxanthin, ceramides, and fatty acids such as omega-7 oil optionally obtained from sea buckthorn.

The disclosed topical compositions preferably are formulated to be supportive and compatible with the skin microbiome and pH. For example, preferably the disclosed topical compositions have a pH within a range of about 6 to about 8. Optionally, the disclosed topical compositions may include a buffering system.

The disclosed topical compositions may be produced by methods known in the art including, but not limited to the following description. This description is not meant to limit future or potential means of production or the claimed subject matter, nor to exhaustively detail all methods currently in use or development. Neither the set of ingredients used in the following description, nor their relative amounts, should be interpreted to limit the claimed subject matter.

A base vehicle comprising honey, shea butter, cocoa butter, and glycerin may be prepared as follows. To an amount of shea butter that consists of 50% of the final formulation by mass, there may be added an amount of softened cocoa butter equal to 25% of the final formulation by mass. After creating a homogenous cream via stirring or blending, an amount of glycerin and honey, equal to 12.5% and 5% of the final formulation by mass, respectively, may be added to the cocoa and shea butter mixture.

A blend containing bacteria from the genera Lactobacillus, Bifidobacterium, or Streptococcus, especially as listed above among, may be dissolved in a solution of distilled water, along with sunflower lecithin, that together are equal to 2.5% of the final formulation by mass. This water-based mixture then may be added to the base vehicle above.

Lastly, sunflower oil and fractioned coconut oil, each in an amount equal to 2.5% of the final formulation by weight, may together be dissolved in the base vehicle above and mixed on low power until integrated to yield a cream-like solution. The resulting product should be refrigerated for 12 to 24 hours. Depending on the crystal structure of the cocoa butter used, applying heat and controlling re-solidification conditions may be necessary to optimize consistency. Preparation may be best conducted slightly above average room temperature, depending on environment and ingredient feedstock. The vehicle and bacterial preparations detailed herein may be adapted for application to other body systems, including for different purposes entirely.

### Illustrative Embodiments

Accordingly, in one embodiment, the composition comprising a bacteria or cell lysate thereof or combination thereof and honey, wherein the bacteria belong to a species selected from Lactobacillus acidophilus, Lactobacillus plantarum, Lactobacillus rhamnosus, Lactobacillus delbruecki, Lactobacillus paracasei, Lactobacillus salivarius, Lactobacillus casei, Bifidobacterium lactis, Bifidobacterium longum, or Bifidobacterium breve, wherein the H. bacteria is killed e.g., by heat treatment.

It will be appreciated by those skilled in the art that any bacteria strain is used.

In some embodiments, the present invention provides strains of Lactobacillus acidophilus having the characteristics of a strain selected from the group consisting of:

| Strain | Assembly No. | Strain | Assembly No. | Strain | Assembly No. |
|---|---|---|---|---|---|
| DSM 20079 | GCA_003047065.1 | KLDS 1.0901 | GCA_001868765.1 | DS20_1 | GCA_003061885.1 |
| YT1 | GCA_003952845.1 | LMG P-21904 | GCA_002914945.1 | LA_AVK2 | GCA_009741835.1 |
| NCFM | GCA_000011985.1 | PNW3 | GCA_004348805.1 | LA_AVK1 | GCA_009742735.1 |
| FSI4 | GCA_000934625.1 | CIRM-BIA 442 | GCA_000442865.1 | UBLA-34 | GCA_003641085.1 |
| ATCC 53544 | GCA_002224305.1 | DSM 20242 | GCA_000442825.1 | DS10_1A | GCA_003053245.1 |
| La-14 | GCA_000389675.2 | DS13_1A | GCA_003061965.1 | BIO6307 | GCA_008868625.1 |
| LA1 | GCA_002286215.1 | DS24_1 | GCA_003053135.1 | DS8_1A | GCA_003061945.1 |
| NBRC 13951 | GCA_001591845.1 | DS11_1A | GCA_003062025.1 | L-55 | GCA_001950045.1 |
| ATCC 4356 | GCA_000786395.1 | DS1_1A | GCA_003062045.1 | MGYG-HGUT-02379 | GCA_902386525.1 |
| JCM 1132 | GCA_000615165.1 | P2 | GCA_002406675.1 | ATCC 4796 | GCA_000159715.1 |
| DSM 20079 | GCA_001433895.1 | DS13_1B | GCA_003061905.1 | DSM 9126 | GCA_000469745.1 |
| CIP 76.13 | GCA_000469705.1 | DS9_1A | GCA_003061925.1 | CIRM-BIA 445 | GCA_000469765.1 |
| DS2_1A | GCA_003062005.1 | DS5_1A | GCA_003061985.1 | WG-LB-IV | GCA_001639165.1 |
| CFH | GCA_000497795.1 | NCTC13721 | GCA_900452495.1 | CFH | GCA_000497815.1 |

In some embodiments, the present invention provides strains of Lactobacillus plantarum having the characteristics of a strain selected from the group consisting:

| Strain | Assembly No. | Strain | Assembly No. | Strain | Assembly No. |
|---|---|---|---|---|---|
| DSM 16365 | GCA_003641165.1 | DS11_9 | GCA_003061805.1 | T10 | GCA_001888275.1 |
| UNQLp11 | GCA_004730965.1 | Kanjika2007 | GCA_001742965.1 | E6-4 | GCA_004300945.1 |
| DF | GCA_001704335.1 | CECT 9571 | GCA_900695365.1 | E6-2 | GCA_004300935.1 |
| KP | GCA_001704315.1 | CECT 9492 | GCA_900695295.1 | Y2-5 | GCA_004301205.1 |
| BDGP2 | GCA_002290185.1 | MHO2.5 | GCA_001888495.1 | E6-6 | GCA_004301035.1 |
| LB1-2 | GCA_002906875.1 | TL2766 | GCA_001675425.1 | RI-265 | GCA_002750695.1 |
| NCU116 | GCA_001672035.1 | P14 | GCA_001888775.1 | E6-1 | GCA_004300925.1 |
| PC520 | GCA_002576835.1 | Lp820 | GCA_002370925.1 | RI-086 | GCA_001981985.1 |
| TMW 1.1478 | GCA_003345375.1 | Lp1610 | GCA_001540925.1 | RI-208 | GCA_001981865.1 |
| dm | GCA_002220175.1 | C4 | GCA_002994725.1 | 4_3 | GCA_000507045.1 |
| 8P-A3 | GCA_009762745.1 | Lp309 | GCA_009889975.1 | CGMCC 8198 | GCA_001754005.1 |
| K259 | GCA_002868775.1 | Nizo2264 | GCA_001639625.1 | FAM 21789 | GCA_005864275.1 |
| 10CH | GCA_002005385.2 | Nizo2263 | GCA_001639595.1 | RI-393 | GCA_002749875.1 |
| WCFS1 | GCA_000203855.3 | NL42 | GCA_000966475.1 | KMB_618 | GCA_003346085.1 |
| SRCM103472 | GCA_004103495.1 | E2C5 | GCA_001597605.1 | Nizo2256 | GCA_001633805.1 |
| SRCM103473 | GCA_004103515.1 | Lp218 | GCA_009890025.1 | RI-405 | GCA_001981785.1 |
| 202195 | GCA_010586945.1 | Lp206 | GCA_009890045.1 | RI-515 | GCA_001982265.1 |
| KC28 | GCA_002948215.1 | DS18_9 | GCA_003053035.1 | RI-048 | GCA_001981585.1 |
| LP2 | GCA_002109425.1 | DS9_9 | GCA_003061785.1 | E6-3 | GCA_004300965.1 |
| B21 | GCA_000931425.2 | DS3_9 | GCA_003053165.1 | PFC-311 | GCA_003046075.1 |
| SRCM103357 | GCA_004101505.1 | Nizo2806 | GCA_001633605.1 | E6-5 | GCA_004300955.1 |
| ZFM55 | GCA_003589725.1 | Nizo2753 | GCA_001633435.1 | RI-513 | GCA_001982385.1 |
| SN35N | GCA_003966855.1 | UBLP-40 | GCA_003692725.1 | Nizo3400 | GCA_001633685.1 |
| JBE245 | GCA_001596095.1 | TMW1.460 | GCA_009864015.1 | Nizol838 | GCA_001639425.1 |
| ZFM9 | GCA_003627335.1 | RI-203 | GCA_002750675.1 | NI326 | GCA_002407395.1 |
| LZ95 | GCA_001484005.1 | L14 | GCA_012070635.1 | Nizo2258 | GCA_001639525.1 |
| LP3 | GCA_002286275.1 | L12 | GCA_011421665.1 | Nizo2257 | GCA_001639505.1 |
| Y44 | GCA_007833595.1 | LpRas | GCA_009889775.1 | WJL | GCA_000474695.1 |
| CAUH2 | GCA_001617525.2 | Nizol837 | GCA_001651845.1 | RI-012 | GCA_001981595.1 |
| ST-III | GCA_000148815.2 | Nizo2260 | GCA_001639565.1 | RI-140 | GCA_001982035.1 |
| SRCM102022 | GCA_002173655.1 | 90sk | GCA_000830535.1 | SRCM101060 | GCA_001662895.1 |
| CACC 558 | GCA_010092485.1 | DSM 20246 | GCA_001888665.1 | P86 | GCA_001888415.1 |
| SRCM102737 | GCA_009913975.1 | E2C2 | GCA_001596195.1 | KMB_621 | GCA_003346075.1 |
| BLS41 | GCA_002116955.1 | DS23_9 | GCA_003053045.1 | M4-2 | GCA_004301025.1 |
| RI-113 | GCA_001990145.1 | DMR 17 | GCA_012272935.1 | YLBGNL-S7 | GCA_006494465.1 |
| IDCC3501 | GCA_003428355.1 | ATCC 8014 | GCA_002631775.1 | Nizo2757 | GCA_001633485.1 |
| KACC 92189 | GCA_003692595.1 | Nizo2741 | GCA_001633415.1 | SF2A35B | GCA_001469145.1 |
| 2-May | GCA_001278015.1 | MHO2.4 | GCA_001888485.1 | M4-4 | GCA_004301075.1 |
| MF1298 | GCA_001880185.2 | Nizo2484 | GCA_001633325.1 | MPL16 | GCA_001660645.1 |
| FBL-3a | GCA_003999275.1 | Nizo2485 | GCA_001633335.1 | NRCC1 | GCA_001649985.1 |
| SK151 | GCA_003269405.1 | Lp201 | GCA_009890095.1 | RI-147 | GCA_001982325.1 |
| LQ80 | GCA_003097595.1 | Lp305 | GCA_009890055.1 | EBKLp545 | GCA_003258615.1 |
| SRCM101167 | GCA_009914095.1 | 8p-a3 | GCA_004403045.1 | EBKLp545 | GCA_009935675.1 |
| IRG1 | GCA_004319665.1 | 8p-a3-Clr | GCA_004404125.1 | RI-165 | GCA_001981575.1 |
| SRCM100440 | GCA_009913635.1 | TISTR875 | GCA_001888725.1 | RI-507 | GCA_001982165.1 |
| SRCM100442 | GCA_009913655.1 | ER | GCA_001633245.1 | CIP104448 | GCA_000956195.1 |
| SRCM100438 | GCA_009913615.1 | DietG20.2.2_EE | GCA_002737995.1 | M4-3 | GCA_004301055.1 |
| SRCM100434 | GCA_002174195.1 | Nizo2535 | GCA_001633405.1 | RI-511 | GCA_001982205.1 |
| TMW 1.1308 | GCA_009619495.1 | Lp90 | GCA_000731855.1 | TJA26B | GCA_003545985.1 |
| ATG-K8 | GCA_003597615.1 | CCFM605 | GCA_003344825.1 | Nizol840 | GCA_001639455.1 |
| 12_3 | GCA_004028335.1 | RI-011 | GCA_001981645.1 | T9 | GCA_004102845.1 |
| SRCM101518 | GCA_009913855.1 | A3 | GCA_001888525.1 | T9 | GCA_004212195.1 |
| ZFM4 | GCA_003627355.1 | Nizo2259 | GCA_001639545.1 | RI-408 | GCA_001981955.1 |
| LZ206 | GCA_001659745.1 | FlyG2.1.8 | GCA_002735815.1 | CECT 8965 | GCA_900290125.1 |
| b-2 | GCA_003352125.1 | UC8491 | GCA_001643065.1 | CECT 8966 | GCA_900290085.1 |
| KCCP11226 | GCA_009720585.1 | Nizo2457 | GCA_001639645.1 | CECT 8963 | GCA_900290135.1 |
| ATG-K6 | GCA_003597595.1 | FlyG20.1.4 | GCA_002737845.1 | Lp790 | GCA_900095045.1 |
| ZJ316 | GCA_000338115.2 | A6 | GCA_002249825.1 | IPLA88 | GCA_000410795.1 |
| ATCC 8014 | GCA_002749655.1 | Nizo2891 | GCA_001633665.1 | RI-510 | GCA_001982195.1 |
| DOMLa | GCA_000604105.1 | IYO1511 | GCA_011170185.1 | Y2-3 | GCA_004301145.1 |
| LM1004 | GCA_002895245.1 | Nizo2889 | GCA_001633675.1 | A3-1 | GCA_004301305.1 |
| BNH17 | GCA_005576935.1 | P42 | GCA_001888335.1 | Y2-1 | GCA_004301135.1 |
| ZS2058 | GCA_001296095.1 | P62 | GCA_001888355.1 | 2025 | GCA_000466905.2 |
| SRCM101105 | GCA_009913695.1 | Lp533 | GCA_009889925.1 | B2-1 | GCA_004301595.1 |
| JDM1 | GCA_000023085.1 | Lp520 | GCA_009889965.1 | R6-2-2 | GCA_004302495.1 |
| DR7 | GCA_003586485.1 | Lp835 | GCA_009889735.1 | A3-2 | GCA_004301325.1 |
| HAC01 | GCA_003143915.1 | Lp510 | GCA_002370985.1 | A4-2 | GCA_004301345.1 |
| CLP0611 | GCA_002024845.1 | Lp542 | GCA_009889895.1 | A1-1 | GCA_004301225.1 |
| LLY-606 | GCA_006770505.1 | Lp541 | GCA_009889935.1 | B1-1 | GCA_004301545.1 |
| TS12 | GCA_001908455.1 | Lp823 | GCA_009889825.1 | TIFN101 | GCA_001005695.1 |
| SRCM100995 | GCA_009913675.1 | Lp543 | GCA_009889885.1 | R14-2-2 | GCA_004302725.1 |
| SRCM103311 | GCA_004101325.1 | P22 | GCA_001888255.1 | wikim18 | GCA_000648755.1 |
| 123-17 | GCA_009759845.1 | Nizo2802 | GCA_001633545.1 | Lp813 | GCA_900095065.1 |
| X7021 | GCA_002943545.1 | JDARSH | GCA_003023825.1 | CECT 8964 | GCA_900290105.1 |
| JBE490 | GCA_002109405.1 | 8 PA 3 | GCA_002234395.1 | A1-2 | GCA_004301245.1 |
| LPL-1 | GCA_002205775.2 | DS14_9 | GCA_003061765.1 | RI-029 | GCA_002751765.1 |
| EM | GCA_004337615.1 | Nizo2494 | GCA_001633355.1 | B2-2 | GCA_004301565.1 |
| SRCM103426 | GCA_004101645.1 | 299v | GCA_001888735.1 | R4-2-2 | GCA_004302295.1 |
| DSR_M2 | GCA_003286955.1 | CIF17A4 | GCA_001888585.1 | A6-1 | GCA_004301425.1 |
| LS/07 | GCA_011304595.1 | CIF17A2 | GCA_001888575.1 | A7-1 | GCA_004301435.1 |
| pc-26 | GCA_006770485.1 | CIF17AN8 | GCA_001888675.1 | A6-2 | GCA_004301445.1 |
| SRCM103300 | GCA_004141875.1 | SKT109 | GCA_004025165.1 | B5-1 | GCA_004301695.1 |
| K25 | GCA_003020005.1 | Nizol839 | GCA_001639445.1 | B4-2 | GCA_004301705.1 |
| KC3 | GCA_002868755.1 | Lp546 | GCA_009889835.1 | B5-2 | GCA_004301725.1 |
| SRCM101222 | GCA_009913835.1 | Lp821 | GCA_009889865.1 | R4-3-2 | GCA_004302285.1 |
| LMT1-48 | GCA_003813125.1 | 02T60C | GCA_002165715.1 | R5-3-2 | GCA_004302405.1 |
| plantarum | GCA_003076435.1 | P31 | GCA_001888325.1 | R14-3-2 | GCA_004302715.1 |
| SRCM103361 | GCA_004101545.1 | RI-422 | GCA_001981875.1 | R14-3-1 | GCA_004302745.1 |
| CGMCC 1.557 | GCA_001272315.2 | BIO1096 | GCA_008868495.1 | R14-2-1 | GCA_004302695.1 |
| TMW 1.25 | GCA_002117245.1 | B-1 | GCA_004122965.1 | B14-1 | GCA_004301815.1 |
| WLPL04 | GCA_001331925.2 | P73 | GCA_001888425.1 | B1-2 | GCA_004301585.1 |
| TMW 1.1623 | GCA_002117305.1 | FlyG3.1.8 | GCA_002738055.1 | B4-1 | GCA_004301685.1 |
| TMW 1.708 | GCA_002117285.1 | JMCC0013 | GCA_002920935.1 | B7-2 | GCA_004301795.1 |
| LZ227 | GCA_001660025.1 | CIF17A5 | GCA_001888595.1 | B7-1 | GCA_004301805.1 |
| nF1-FD | GCA_003952885.1 | ZPZ | GCA_008016415.1 | B14-2 | GCA_004301865.1 |
| nF1 | GCA_003325395.1 | FlyG11.1.2 | GCA_002737975.1 | R3-2-2 | GCA_004302185.1 |
| LY-78 | GCA_001715615.1 | DietG20.1.2 | GCA_002737935.1 | R14-1-2 | GCA_004302685.1 |
| SRCM103418 | GCA_004101625.1 | L31-1 | GCA_001267905.1 | R3-3-2 | GCA_004302215.1 |
| SRCM103303 | GCA_004141895.1 | FlyG8.1.1 | GCA_002737745.1 | R6-2-1 | GCA_004302485.1 |
| SRCM103295 | GCA_004087995.1 | RI-146 | GCA_001982305.1 | A4-1 | GCA_004301335.1 |
| TMW 1.277 | GCA_002117265.1 | P76 | GCA_001888465.1 | A5-1 | GCA_004301355.1 |
| SRCM103297 | GCA_004141755.1 | PC520 | GCA_001704595.1 | A7-2 | GCA_004301455.1 |
| C410L1 | GCA_001874125.1 | 1 | GCA_900080205.1 | R1-1-1 | GCA_004301875.1 |
| X7022 | GCA_011022295.1 | CGMCC12436 | GCA_003344845.1 | R3-1-1 | GCA_004302115.1 |
| 83-18 | GCA_009759825.1 | EGD-AQ4 | GCA_000463075.2 | R3-2-1 | GCA_004302135.1 |
| SRCM101511 | GCA_009937825.1 | Lp998 | GCA_900095055.1 | R5-2-2 | GCA_004302395.1 |
| HFC8 | GCA_001302645.1 | INF 15D | GCA_004368485.1 | R6-3-1 | GCA_004302515.1 |
| 16 | GCA_000412205.1 | RI-162 | GCA_001981565.1 | R2-3-2 | GCA_004302105.1 |
| GB-LP1 | GCA_002220815.1 | XZ3303 | GCA_004123095.1 | A2-1 | GCA_004301235.1 |
| SRCM103362 | GCA_004101605.1 | I08 | GCA_001888565.1 | A5-2 | GCA_004301395.1 |
| SPC-SNU 72-2 | GCA_012109355.1 | S11T3E | GCA_002165725.1 | A14-1 | GCA_004301485.1 |
| plantarum | GCA_000392485.2 | MHO2.9 | GCA_001888505.1 | A14-2 | GCA_004301555.1 |
| ATG-K2 | GCA_003597635.1 | dkp1 | GCA_004118615.1 | B3-2 | GCA_004301655.1 |
| NCIMB 700965 | GCA_003611015.1 | 49 | GCA_003347445.1 | B6-1 | GCA_004301765.1 |
| NCIMB700965.EF.A | GCA_004328745.1 | Curd | GCA_003990985.1 | R3-1-2 | GCA_004302125.1 |
| SRCM101187 | GCA_009913795.1 | CECT 9491 | GCA_902825385.1 | R5-1-2 | GCA_004302345.1 |
| YW11 | GCA_004028295.1 | FlyG8.1.2 | GCA_002737775.1 | R5-3-1 | GCA_004302415.1 |
| 13_3 | GCA_004028315.1 | FlyG10.1.5 | GCA_002737835.1 | R6-1-2 | GCA_004302505.1 |
| Zhang-LL | GCA_001581895.1 | FlyG11.1.6 | GCA_002737965.1 | R7-2-1 | GCA_004302615.1 |
| Q7 | GCA_003999605.1 | FBR6 | GCA_001619295.1 | R2-2-2 | GCA_004302055.1 |
| El | GCA_003429585.1 | KMB_597 | GCA_003346175.1 | B3-1 | GCA_004301645.1 |
| AS-8 | GCA_003045665.1 | K35 | GCA_001888265.1 | B6-2 | GCA_004301775.1 |
| AS-6 | GCA_003045725.1 | D13 | GCA_002532115.1 | R3-3-1 | GCA_004302195.1 |
| AS-10 | GCA_003045645.1 | Nizo2801 | GCA_001633565.1 | M4-1 | GCA_004301045.1 |
| AS-9 | GCA_003045705.1 | AM25-20AC | GCA_003470765.1 | R6-3-2 | GCA_004302535.1 |
| CMPG5300 | GCA_000762955.1 | FlyG10.1.9 | GCA_002738045.1 | 2003wt genome | GCA_900200135.1 |
| | | | | assembly | |
| ATCC 14917 | GCA_000143745.1 | DS6_9 | GCA_003061725.1 | NBRC 106468 | GCA_007991855.1 |
| CGMCC 1.2437 | GCA_001434175.1 | LR46 | GCA_009807195.1 | R4-1-1 | GCA_004302205.1 |
| NBRC 15891 | GCA_007989145.1 | LR39 | GCA_009807205.1 | R14-1-1 | GCA_004302645.1 |
| DSM 13273 | GCA_001436855.1 | Nizo2814 | GCA_001633575.1 | R2-1-2 | GCA_004302005.1 |
| JSA22 | GCA_001720285.1 | DmCS_001 | GCA_000743895.1 | A2-2 | GCA_004301255.1 |
| C29 | GCA_003577505.1 | CNW10 | GCA_001633285.1 | R2-2-1 | GCA_004302035.1 |
| MGYG-HGUT-02386 | GCA_902386645.1 | 161 | GCA_001888245.1 | R4-2-1 | GCA_004302255.1 |
| SRCM103406 | GCA_004078535.1 | BFE 5092 | GCA_900078525.1 | R6-1-1 | GCA_004302445.1 |
| SRCM103411 | GCA_004078645.1 | FlyG7.1.6 | GCA_002738065.1 | 43-3 | GCA_001595615.1 |
| BK-022 | GCA_009756965.1 | LR14 | GCA_009807215.1 | AM25-25 | GCA_003469805.1 |
| SRCM103305 | GCA_004055415.1 | KB1253 | GCA_004000705.1 | FUA3590 | GCA_004683785.1 |
| SRCM103292 | GCA_004055435.1 | AMBR9 | GCA_901830435.1 | Nizo2776 | GCA_001633455.1 |
| SRCM103287 | GCA_004054305.1 | SNU.Lpl77 | GCA_001273585.1 | R4-3-1 | GCA_004302305.1 |
| PS128 | GCA_001005805.1 | FBR5 | GCA_001619265.1 | R1-3-2 | GCA_004301975.1 |
| Nizo2877 | GCA_001308305.1 | P26 | GCA_001888345.1 | R5-1-1 | GCA_004302315.1 |
| AG30 | GCA_000687495.1 | RI-266 | GCA_001982125.1 | R7-1-1 | GCA_004302585.1 |
| CRL 1506 | GCA_001444495.1 | 201 | GCA_003347455.1 | R1-1-2 | GCA_004301885.1 |
| WJL | GCA_001307325.1 | FlyG9.1.4 | GCA_002737825.1 | R5-2-1 | GCA_004302385.1 |
| LMG S-29189 | GCA_002914965.1 | CIF17AN2 | GCA_001888645.1 | R2-1-1 | GCA_004301995.1 |
| | GCA_000247735.2 | CECT 9434 | GCA_900695425.1 | R2-3-1 | GCA_004302065.1 |
| FMNP01 | GCA_000764285.1 | CECT 9435 | GCA_900700275.1 | IMAU80873 | GCA_009766195.1 |
| SRCM101520 | GCA_002872355.1 | FlyG9.2.5 | GCA_002737925.1 | LP91 | GCA_000473935.1 |
| SF9C | GCA_009914805.1 | CECT 8962 | GCA_900289155.1 | R1-3-1 | GCA_004301955.1 |
| FlyG11.2.6 | GCA_002737905.1 | RI-514 | GCA_001982405.1 | Y2-2 | GCA_004301125.1 |
| FUA3584 | GCA_009863935.1 | SF15C | GCA_002532125.1 | KMB_619 | GCA_003346235.1 |
| Nizo3892 | GCA_001633765.1 | EML1 | GCA_008016845.1 | RI-512 | GCA_001982245.1 |
| FlyG20.2.6 | GCA_002737885.1 | SA3 | GCA_008016855.1 | 19L3 | GCA_000604365.1 |
| CRL 681 | GCA_003325775.1 | UCMA3037 | GCA_000347515.1 | R1-2-1 | GCA_004301915.1 |
| K36 | GCA_001888745.1 | Tw226 | GCA_011040375.1 | L. plantarum A6 | GCA_900176235.1 |
| Nizo2726 | GCA_001633385.1 | S2T10D | GCA_002165655.1 | R7-2-2 | GCA_004302625.1 |
| Nizo2855 | GCA_001633645.1 | Nizo3893 | GCA_001633745.1 | LL441 | GCA_001754025.1 |
| ATCC 8014 | GCA_002370965.1 | RI-505 | GCA_001982345.1 | IMAU20970 | GCA_009766165.1 |
| DS8_9 | GCA_003053025.1 | L10 | GCA_009295775.1 | Y2-4 | GCA_004301155.1 |
| 8 RA-3 | GCA_001010175.1 | P67 | GCA_001888405.1 | M92C | GCA_002532175.1 |
| P5 | GCA_011009755.1 | FBR4 | GCA_001619275.1 | R1-2-2 | GCA_004301905.1 |
| DSM 2601 | GCA_001888655.1 | C1 | GCA_008016925.1 | KLDS1.0391 | GCA_002028365.1 |
| NTTN08 | GCA_006364975.1 | Nizo2766 | GCA_001633495.1 | NCTC13644 | GCA_900452585.1 |
| Nizo2830 | GCA_001633505.1 | Nizo2029 | GCA_001639485.1 | 19.1 | GCA_001633265.1 |
| Nizo2831 | GCA_001633595.1 | RI-509 | GCA_001982335.1 | JCM 1149 | GCA_000615325.1 |
| D31 | GCA_004102885.1 | 80 | GCA_001368775.1 | NAB2 | GCA_001633255.1 |
| Nizo3894 | GCA_001633725.1 | YW32 | GCA_004123035.1 | NAB1 | GCA_001633775.1 |
| Nizo2262 | GCA_001639585.1 | RI-123 | GCA_001982005.1 | DSM 16365 | GCA_001435215.1 |
| ATCC 202195 | GCA_004354995.1 | KMB_614 | GCA_003346105.1 | SRCM101258 | GCA_002906095.1 |
| Lp519 | GCA_009889995.1 | RI-139 | GCA_001982285.1 | WHE 92 | GCA_000604145.1 |
| Lpl612 | GCA_001540965.1 | RI-189 | GCA_001981665.1 | 2165 | GCA_000466845.1 |
| XJ25 | GCA_001704645.1 | RI-191 | GCA_002750575.1 | UBA4900 | GCA_002398245.1 |
| DS13_9 | GCA_003053185.1 | RI-506 | GCA_001982115.1 | 38 | GCA_001010255.1 |
| J26 | GCA_004771035.1 | NF92 | GCA_003709415.1 | RI-190 | GCA_001981655.1 |

In some embodiments, the present invention provides strains of Lactobacillus rhamnosus having the characteristics of a strain selected from the group consisting:

| Strain | Assembly | Strain | Assembly | Strain | Assembly |
|---|---|---|---|---|---|
| GG (ATCC 53103) | GCA_000026505.1 | BIOML-A8 | GCA_009679295.1 | Lrh32 | GCA_001656545.1 |
| NCTC13764 | GCA_900636965.1 | Lrh11 | GCA_001656735.1 | Lrh8 | GCA_001656535.1 |
| BFE5264 | GCA_001988935.1 | Lrh20 | GCA_001656675.1 | Lrh19 | GCA_001656685.1 |
| 4B15 | GCA_002158925.1 | INIA P344 | GCA_901971785.1 | Lrh17 | GCA_001657075.1 |
| BPL5 | GCA_900070175.1 | BIOML-A2 | GCA_009679405.1 | 116 | GCA_000801045.1 |
| hsryfm 1301 | GCA_008727835.1 | Vahe | GCA_008017355.1 | LMS2-1 | GCA_000160175.1 |
| DSM 14870 | GCA_002287945.1 | BIOML-A7 | GCA_009679305.1 | Lrh9 | GCA_001656785.1 |
| GG | GCA_003353455.1 | AMBR1 | GCA_901830405.1 | Lrh34 | GCA_001656765.1 |
| MGYG-HGUT-01293 | GCA_902381635.1 | Lrh31 | GCA_001656575.1 | Lrh25 | GCA_001656975.1 |
| LR-B1 | GCA_004010975.1 | Lrh22 | GCA_001656655.1 | LR-S | GCA_004125475.1 |
| BIO6870 | GCA_008831425.1 | L35 | GCA_000784395.1 | Lrh15 | GCA_001656715.1 |
| ATCC 53103 | GCA_000011045.1 | L34 | GCA_000784375.1 | ATCC 21052 | GCA_000235865.1 |
| NCTC13710 | GCA_900636875.1 | LR-CVC | GCA_004125455.1 | 784_LRHA | GCA_001067335.1 |
| LOCK908 | GCA_000418495.1 | LR-B2 | GCA_004125395.1 | ASCC 1521 | GCA_001831275.1 |
| ATCC 11443 | GCA_003433395.1 | BIOML-A5 | GCA_009679345.1 | ASCC 3018 | GCA_001831215.1 |
| SCT-10-10-60 | GCA_002960215.1 | 313 | GCA_001044415.1 | Lrh16 | GCA_001657085.1 |
| BIO5326 | GCA_009720565.1 | L156.4 | GCA_001991035.1 | Lrh6 | GCA_001656835.1 |
| LR5 | GCA_002286235.1 | AMC143 | GCA_001982425.1 | Lr108 | GCA_001044025.1 |
| Lc 705 | GCA_000026525.1 | IBL027 | GCA_002238035.1 | Lrh5 | GCA_001656845.1 |
| ATCC 8530 | GCA_000233755.1 | 40f | GCA_001044405.1 | 893_LRHA | GCA_001067625.1 |
| 1.032 | GCA_006151905.1 | INIA P540 | GCA_901971795.1 | 944_LRHA | GCA_001068215.1 |
| LRB | GCA_001721925.1 | DS4_11 | GCA_003052965.1 | 988_LRHA | GCA_001068045.1 |
| Pen | GCA_002076955.1 | AMC010 | GCA_001982435.1 | 769_LRHA | GCA_001067215.1 |
| LOCK900 | GCA_000418475.1 | Lrh7 | GCA_001656815.1 | 708_LRHA | GCA_001067025.1 |
| IDCC 3201 | GCA_009429065.1 | BPL15 | GCA_001368735.1 | 943_LRHA | GCA_001068195.1 |
| ASCC 290 | GCA_001590655.1 | PEL5 | GCA_000712505.1 | LR231 | GCA_000508405.1 |
| WQ2 | GCA_002025085.1 | Lrh46 | GCA_002103215.1 | 906_LRHA | GCA_001067885.1 |
| NRRL B-442 | GCA_002849515.1 | Lrh23 | GCA_001656635.1 | 699_LRHA | GCA_001066975.1 |
| DSM 20021 | GCA_001435405.1 | Lrh39 | GCA_002103145.1 | Lrh33 | GCA_001657195.1 |
| NBRC 3425 | GCA_007990855.1 | Lrh21 | GCA_001656995.1 | ASCC 3016 | GCA_001831225.1 |
| DS17_11 | GCA_003061565.1 | Lrhl4 | GCA_001657115.1 | 870_LRHA | GCA_001066715.1 |
| R0011 | GCA_000235785.2 | L31 | GCA_000784405.1 | 979_LRHA | GCA_001068015.1 |
| DS22_11 | GCA_003061605.1 | CLS17 | GCA_000932035.1 | Lrh4 | GCA_001656875.1 |
| K32 | GCA_000735255.1 | P5 | GCA_002406715.1 | 214_LRHA | GCA_001062955.1 |
| CNCM-I-3698 | GCA_001005625.1 | P4 | GCA_002406745.1 | Lrh24 | GCA_001657005.1 |
| 51B | GCA_000699985.1 | P1 | GCA_002406795.1 | Lrh3 | GCA_001656915.1 |
| GR-1 | GCA_002762445.1 | R19-3 | GCA_001645615.1 | Lactobacillus | GCA_900604925.1 |
| | | | | rhamnosus GR-1 | |
| UBLR-58 | GCA_004798455.1 | ASCC 3029 | GCA_001831235.1 | Lrh12 | GCA_001657155.1 |
| B1 | GCA_002406705.1 | HCT70 | GCA_001756565.1 | Lrh2 | GCA_001657025.1 |
| LR_AVK | GCA_009742715.1 | E800 | GCA_000712495.1 | 390_LRHA | GCA_001064785.1 |
| BIOML-A3 | GCA_009679395.1 | Lrhl3 | GCA_001657135.1 | Lrh29 | GCA_001656585.1 |
| DS12_11 | GCA_003061665.1 | Lrh28 | GCA_001656925.1 | Lactobacillus | GCA_900248175.2 |
| | | | | rhamnosus | |
| BIOML-A4 | GCA_009679335.1 | Lrhl8 | GCA_001657055.1 | CASL | GCA_000226235.1 |
| FAM 20558 | GCA_005864245.1 | Lrh26 | GCA_001656605.1 | Lrh1 | GCA_001656755.1 |
| DS18_11 | GCA_003052925.1 | Lrh42 | GCA_001657205.1 | Lrh43 | GCA_001657255.1 |
| DS3_11 | GCA_003052985.1 | 526_LRHA | GCA_001063655.1 | Lrl38 | GCA_001044075.1 |
| DS9_11 | GCA_003061625.1 | 541_LRHA | GCA_001065365.1 | Lr071 | GCA_001044015.1 |
| Lrh47 | GCA_002103185.1 | 308 | GCA_000814485.1 | Lrh44 | GCA_001657235.1 |
| LRHMDP3 | GCA_000311965.1 | Lrh10 | GCA_001657165.1 | Lr073 | GCA_001043995.1 |
| Lrh40 | GCA_009805825.1 | LR-GG-MoProbi | GCA_004125465.1 | BIOML-A1 | GCA_009679445.1 |
| LRHMDP2 | GCA_000311945.1 | PEL6 | GCA_000712515.1 | AMBR4 | GCA_901830395.1 |
| R709 | GCA_002027355.1 | Lrh30 | GCA_001656895.1 | CRL1505 | GCA_000414365.1 |
| LR863 | GCA_003129645.1 | 24 | GCA_000743075.1 | NRRL B-442 | GCA_003352825.1 |
| TMC3115 | GCA_003129615.1 | HN001 | GCA_000173255.2 | AMBR3 | GCA_901830385.1 |
| ARJD | GCA_003573615.1 | Lrh38 | GCA_002103155.1 | P3 | GCA_002406785.1 |
| 319_LRHA | GCA_001064515.1 | Lrh27 | GCA_001656945.1 | JCM 1136 | GCA_000615245.1 |
| UMB0004 | GCA_002848015.1 | 186_LRHA | GCA_001062885.1 | 2166 | GCA_000466865.2 |
| DS13_11 | GCA_003052945.1 | RI-004 | GCA_001981725.1 | AMBR5 | GCA_901830425.1 |
| BIOML-A6 | GCA_009679355.1 | DS15_11 | GCA_003061645.1 | AMBR6 | GCA_901830355.1 |
| LR2 | GCA_003046115.1 | DS14_11 | GCA_003061705.1 | AMBR7 | GCA_901830365.1 |
| 43952 | GCA_004122925.1 | co_0103 | GCA_004167055.1 | 389_LRHA | GCA_001063295.1 |
| 4928STDY7387919 | GCA_902166035.1 | BIOML-A10 | GCA_009679255.1 | Lr053 | GCA_001044085.1 |
| Lr044 | GCA_001044105.1 | BIOML-A9 | GCA_009679265.1 | Lr032 | GCA_001044095.1 |
| Lr140 | GCA_001044005.1 | MTCC 5462 | GCA_000195375.2 | Lrh45 | GCA_001657245.1 |

In some embodiments, the present invention provides strains of Lactobacillus delbruecki having the characteristics of a strain selected from the group consisting:

| Strain | Assembly | Strain | Assembly | Strain | Assembly |
|---|---|---|---|---|---|
| ATCC 11842 | GCA_000056065.1 | JCM 17838 | GCA_001190005.1 | PB2003/044-T3-4 | GCA_000179375.1 |
| DSM 20072 | GCA_002278095.1 | DSM | GCA_004354615.1 | FAM 21277 | GCA_005864055.1 |
| | | 26046 | | | |
| JCM 17838 | GCA_001888965.1 | ZN7a-9 | GCA_000387565.1 | KACC 13439 | GCA_001263315.1 |
| JCM 15610 | GCA_001908415.1 | DSM 26046 | GCA_001437485.1 | MGYG-HGUT-01369 | GCA_902374335.1 |
| NBRC 3202 | GCA_006740305.1 | JCM 15610 | GCA_001189855.1 | | GCA_000751695.2 |
| DSM 26046 | GCA_001888925.1 | DSM 15996 | GCA_001435795.1 | NBRC 3734 | GCA_006538685.1 |
| KCCM 34717 | GCA_001888905.1 | NBRC 13953 | GCA_006539405.1 | CRL581 | GCA_000409675.1 |
| KCTC 3034 | GCA_002285775.1 | DSM 20081 | GCA_001437195.1 | | GCA_000751275.1 |
| ND02 | GCA_000182835.1 | DSM 20072 | GCA_000192165.1 | NWC_1_2 | GCA_003814285.1 |
| Lactobacillus | GCA_900322585.1 | DSM 20072 | GCA_001434635.1 | DSM 20072 | GCA_002017855.1 |
| delbrueckii | | | | | |
| subsp. lactis1 | | | | | |
| TUA4408L | GCA_002142575.1 | DSM 20074 | GCA_001433875.1 | Lacto_22L | GCA_900088455.1 |
| KCTC 3035 | GCA_001888985.1 | KCTC 3034 | GCA_002016675.1 | CRL871 | GCA_000934805.1 |
| KCTC 13731 | GCA_001888945.1 | | GCA_000751655.1 | JCM 1002 | GCA_001311275.1 |
| LJJ | GCA_011044195.1 | | GCA_000751895.1 | NBRC3202 | GCA_002723875.1 |
| KLDS1.1011 | GCA_006704185.1 | | GCA_000751635.1 | JCM 1012 | GCA_000615095.1 |
| MN-BM-F01 | GCA_001469775.1 | CFL1 | GCA_001510975.1 | W1P13.016 | GCA_004556255.1 |
| 2038 | GCA_000191165.1 | TJA31 | GCA_003545935.1 | KW14_3 | GCA_004123065.1 |
| KLDS1.0207 | GCA_003597655.1 | MBT 92059 | GCA_003326615.1 | NBRC 3376 | GCA_006538625.1 |
| DSM 20080 | GCA_001953135.1 | Lb1-WT | GCA_001624905.1 | | GCA_000751235.1 |
| ND04 | GCA_002000885.1 | Lb1-GS-1 | GCA_001624925.1 | UMB0003 | GCA_002847905.1 |
| ATCC BAA-365 | GCA_000014405.1 | IAHAHI | GCA_008016355.1 | LBB.B5 | GCA_001647065.1 |
| Lactobacillus | GCA_900196735.1 | AVK | GCA_003034905.1 | LDELB18P2 | GCA_004211735.1 |
| bulgaricus | | | | | |
| ACA-DC 87 | | | | | |
| TS1-06 | GCA_009734125.1 | CNCM I-1632 | GCA_000284695.1 | NBRC 3534 | GCA_006538645.1 |
| L99 | GCA_003351805.1 | 328M | GCA_009362855.1 | UBLB01 | GCA_008014735.1 |
| DSM 20074 | GCA_001908495.1 | CNCM I-1519 | GCA_000284715.1 | LDELB18P1 | GCA_004211505.1 |
| YNF-5 | GCA_004122955.1 | | | FAM 21784 | GCA_005864125.1 |

In some embodiments, the present invention provides strains of Lactobacillus paracasei having the characteristics of a strain selected from the group consisting:

| Strain | Assembly | Strain | Assembly | Strain | Assembly |
|---|---|---|---|---|---|
| JCM 8130 | GCA_000829035.1 | UBLPC-35 | GCA_003640765.1 | FAM6012 | GCA_003712745.1 |
| IBB3423 | GCA_009739485.1 | T71499 | GCA_000309685.1 | FAM18172 | GCA_003712585.1 |
| CACC 566 | GCA_009931715.1 | FAM19353 | GCA_003712605.1 | UCD174 | GCA_000309705.1 |
| LOCK919 | GCA_000418515.1 | RI-210 | GCA_001981715.1 | FAM22279 | GCA_003712705.1 |
| IJH-SONE68 | GCA_003966835.1 | Lpp225 | GCA_000410175.1 | FAM18105 | GCA_003712275.1 |
| NJ | GCA_007637635.1 | CRF28 | GCA_000309645.1 | Lpp17 | GCA_000410135.1 |
| SRCM103299 | GCA_004141835.1 | FAM6410 | GCA_003712965.1 | FAM18157 | GCA_003712865.1 |
| TCS | GCA_008807095.1 | VA02-1AN | GCA_011031945.1 | FAM18110 | GCA_003712265.1 |
| L9 | GCA_001244395.1 | L32 | GCA_011421685.1 | D10-4 | GCA_010363715.1 |
| MGYG-HGUT-02388 | GCA_902386635.1 | FAM19317 | GCA_003712565.1 | FAM18126 | GCA_003712925.1 |
| BD-II | GCA_000194765.1 | LactoQK | GCA_008904885.1 | TJB4 | GCA_003545945.1 |
| TMW 1.1434 | GCA_002813615.1 | FAM6161 | GCA_003712785.1 | KL1-Liu | GCA_000827145.1 |
| IIA | GCA_002079285.1 | FAM18123 | GCA_003712325.1 | NRIC0644 | GCA_000958525.1 |
| Lpc10 | GCA_003199005.1 | LC-Ikematsu | GCA_001895185.1 | Lpp223 | GCA_000409955.1 |
| LC355 | GCA_003268715.1 | Lpl7 | GCA_000409835.1 | Lpp230 | GCA_000409815.1 |
| HD1.7 | GCA_002865565.1 | Lc1542 | GCA_001540885.1 | Lppl22 | GCA_000409855.1 |
| LC2W | GCA_000194785.1 | DPC6800 | GCA_001469115.1 | Lpl14 | GCA_000410335.1 |
| HDS-01 | GCA_002902825.1 | DTA83 | GCA_003571925.1 | FAM22280 | GCA_003712725.1 |
| CBA3611 | GCA_007292115.1 | Lc-10 | GCA_000309765.1 | FAM6165 | GCA_003712875.1 |
| 7112-2 | GCA_003957435.1 | INIA P272 | GCA_901933195.1 | KMB_622 | GCA_003346025.1 |
| ZFM54 | GCA_003627255.1 | SA5 | GCA_008016825.1 | FAM18113 | GCA_003712395.1 |
| 10266 | GCA_008329845.1 | CCC B1205 | GCA_002904305.1 | FAM18168 | GCA_003712505.1 |
| TK1501 | GCA_002257625.1 | D3-5 | GCA_009996805.1 | UW4 | GCA_000376145.1 |
| 8700:02:00 | GCA_000155515.2 | FAM18175 | GCA_003712535.1 | INF 448 | GCA_004368425.1 |
| N1115 | GCA_000582665.1 | FAM22276 | GCA_003712645.1 | 1316.rep1_LPAR | GCA_001062665.1 |
| EG9 | GCA_003177075.1 | FAM3248 | GCA_003712935.1 | RI-195 | GCA_001982095.1 |
| KL1 | GCA_001514415.1 | S49 | GCA_005049135.1 | UW1 | GCA_000309725.1 |
| ATCC 334 | GCA_000014525.1 | M36 | GCA_000309665.1 | Lpp74 | GCA_000410235.1 |
| Zhang | GCA_000019245.3 | FAM19404 | GCA_003712625.1 | FAM18133 | GCA_003712525.1 |
| CAUH35 | GCA_001191565.1 | DSM 20207 | GCA_000949485.1 | COM0101 | GCA_000508845.1 |
| FAM18149 | GCA_002442835.1 | AM33-2AC | GCA_003434205.1 | INF 10 | GCA_004368475.1 |
| TD 062 | GCA_009834405.1 | KMB_598 | GCA_003367655.1 | Lpp43 | GCA_000410455.1 |
| LcY | GCA_000388095.2 | DmW_181 | GCA_002115655.1 | Lppl25 | GCA_000410475.1 |
| DSM 5622 | GCA_001436385.1 | NRIC1917 | GCA_000958505.1 | FAM22278 | GCA_003712685.1 |
| ATCC 25302 | GCA_004354655.1 | Lpp226 | GCA_000409895.1 | 844_LCAS | GCA_001066565.1 |
| NBRC 15889 | GCA_007989125.1 | Lpc-37 | GCA_000309785.1 | KMB_616 | GCA_003346265.1 |
| ATCC 25302 | GCA_000159495.1 | 525_LPAR | GCA_001076935.1 | BIOML-A4 | GCA_009679455.1 |
| DSM 20258 | GCA_001436485.1 | FAM8407 | GCA_003712835.1 | INF 456 | GCA_004368495.1 |
| NBRC 15906 | GCA_007989185.1 | DTA93 | GCA_008369945.1 | Lpp22 | GCA_000410155.1 |
| Lpc-37 | GCA_002762235.1 | BIOML-A1 | GCA_009679505.1 | Lpp228 | GCA_000409995.1 |
| SRCM103424 | GCA_004078565.1 | RI-194 | GCA_001982085.1 | Lpp219 | GCA_000410195.1 |
| JS1 | GCA_003795255.1 | Lpp46 | GCA_000409875.1 | KMB_623 | GCA_003346015.1 |
| SRCM103410 | GCA_004078555.1 | Lpp49 | GCA_000410035.1 | Lpp221 | GCA_000410015.1 |
| FAM3228 | GCA_003712905.1 | DTA72 | GCA_009823605.1 | 1316.rep2_LPAR | GCA_001062695.1 |
| FAM18099 | GCA_003712295.1 | BIOML-A3 | GCA_009679435.1 | Lpp37 | GCA_000410415.1 |
| FAM18101 | GCA_003712355.1 | L9D | GCA_001858275.1 | Lppl89 | GCA_000410295.1 |
| FAM18121 | GCA_003712345.1 | FAM18132 | GCA_003712455.1 | NRIC1981 | GCA_000958485.1 |
| FAM10859 | GCA_003712225.1 | co_0103 | GCA_004167215.1 | Lpp229 | GCA_000410215.1 |
| FAM18149 | GCA_003712485.1 | BIOML-A2 | GCA_009679495.1 | CNCM I-4270 | GCA_000410275.1 |
| FAM3257 | GCA_003712755.1 | 275_LPAR | GCA_001076595.1 | BM-LC14617 | GCA_001636215.1 |
| FAM 3257 | GCA_005864195.1 | FAM18124 | GCA_003712425.1 | 12A | GCA_000472345.1 |
| DPC2071 | GCA_002148875.1 | FAM18129 | GCA_003712445.1 | Lppl4 | GCA_000410315.1 |
| W14 | GCA_002027415.1 | W16 | GCA_002027405.1 | Lbs2 | GCA_000736295.5 |
| FAM22277 | GCA_003712665.1 | FAM18119 | GCA_003712385.1 | CNCM I-2877 | GCA_000410095.1 |
| FAM4067 | GCA_003712795.1 | HZ-1 | GCA_002120125.1 | Lpp70 | GCA_000410495.1 |
| DPC4536 | GCA_002148885.1 | Lpp120 | GCA_000409935.1 | Lpp7 | GCA_000410255.1 |
| BIO5452 | GCA_008868525.1 | NTU 101 | GCA_002901165.1 | Lpp227 | GCA_000410055.1 |
| DTA81 | GCA_012241605.1 | Lpp71 | GCA_000410375.1 | LcA | GCA_000400585.1 |
| DUP 13076 | GCA_002849575.1 | Lpp41 | GCA_000410395.1 | 32G | GCA_000309605.1 |
| LMG S-29188 | GCA_002914885.1 | Lpp48 | GCA_000410115.1 | B2 | GCA_002406765.1 |
| DPC4206 | GCA_002148805.1 | Lppl23 | GCA_000409975.1 | CNCM I-4649 | GCA_000410435.1 |
| FAM18108 | GCA_003712245.1 | Lppl26 | GCA_000410355.1 | CNCM I-4648 | GCA_000410075.1 |
| FAM8374 | GCA_003712825.1 | FAM8140 | GCA_003712985.1 | 5b | GCA_000474615.1 |
| FAM7821 | GCA_003713005.1 | B3 | GCA_002406665.1 | | |

In some embodiments, the present invention provides strains of Lactobacillus salivarius having the characteristics of a strain selected from the group consisting:

| Assembly No. | Assembly No. | Assembly No. | Assembly No. |
|---|---|---|---|
| NZ_LT604074 | NZ_NBEO00000000 | NZ_CP007646 | NZ_LXZQ00000000 |
| NZ_NFHV00000000 | NZ_CP017108 | NZ_VSUO00000000 | NZ_LXZP00000000 |
| NZ_VCMN00000000 | NZ_VSVV00000000 | NZ_VSUP00000000 | NZ_NDYW00000000 |
| NZ_VCMO00000000 | NZ_CP017109 | NZ_VSUN00000000 | NZ_VSUJ00000000 |
| NZ_NBEU00000000 | NZ_CP017110 | NZ_VSUM00000000 | NZ_VSUI00000000 |
| NZ_NBET00000000 | NZ_CP029616 | NZ_VSUL00000000 | NZ_VSUH00000000 |
| NZ_NBES00000000 | NZ_VSVI00000000 | NZ_VSTT00000000 | NZ_VSUG00000000 |
| NZ_NBER00000000 | NZ_QAZG00000000 | NZ_VSUD00000000 | NZ_VSUF00000000 |
| NZ_NBEQ00000000 | NZ_QAZF00000000 | NZ_VSUC00000000 | NZ_VSTL00000000 |
| NZ_NBEP00000000 | NZ_NBEN00000000 | NZ_VSUB00000000 | NZ_LXYY00000000 |
| NZ_JUQA00000000 | NZ_NBEM00000000 | NZ_VSUA00000000 | NZ_LXYX00000000 |
| NZ_CP024069 | NZ_VSWA00000000 | NZ_VSTZ00000000 | NZ_NBED00000000 |
| NZ_LT604075 | NZ_VSVZ00000000 | NZ_VSTY00000000 | NZ_VSTS00000000 |
| NZ_CABMGV00000000 | NZ_VSVY00000000 | NZ_VSTX00000000 | NZ_NBEC00000000 |
| NZ_MSCR00000000 | NZ_QAGU00000000 | NZ_VSTW00000000 | NZ_NBEB00000000 |
| NZ_PKGN00000000 | NZ_VSVX00000000 | NZ_VSTV00000000 | NZ_NBEA00000000 |
| NZ_JVAF00000000 | NZ_VSVS00000000 | NZ_VSTU00000000 | NZ_NBDZ00000000 |
| NZ_VSWB00000000 | NZ_NBEX00000000 | NZ_VSUK00000000 | NZ_NBDY00000000 |
| NZ_JUTI00000000 | NZ_NBEW00000000 | NZ_VSUE00000000 | NZ_NBDX00000000 |
| NZ_VSVE00000000 | NZ_NBEV00000000 | NZ_VSTR00000000 | NZ_VSTM00000000 |
| NZ_QFAS00000000 | NZ_QRMK00000000 | NZ_VSUW00000000 | NZ_LXYZ00000000 |
| NZ_LXZB00000000 | NZ_QSLH00000000 | NZ_VSTQ00000000 | NZ_VSTK00000000 |
| NZ_VSVD00000000 | NZ_NFLK00000000 | NZ_VSTP00000000 | NZ_AEBA00000000 |
| NZ_LXZL00000000 | NZ_VSVM00000000 | NZ_VSTO00000000 | NZ_AEBA00000000 |
| NZ_LXZK00000000 | NZ_VSVU00000000 | NZ_NBEJ00000000 | NZ_ACGT00000000 |
| NZ_LXZJ00000000 | NZ_VSVT00000000 | NZ_NBEI00000000 | NZ_ACGT00000000 |
| NZ_LXZH00000000 | NZ_LXZO00000000 | NZ_NBEH00000000 | NZ_AYYT00000000 |
| NZ_LXZG00000000 | NZ_VSVR00000000 | NZ_CP007650 | NZ_AFOI00000000 |
| NZ_LXZF00000000 | NZ_VSVQ00000000 | NZ_VSTI00000000 | NZ_AFOI00000000 |
| NZ_LXZE00000000 | NZ_VSVP00000000 | NZ_QSTB00000000 | NZ_AFMN00000000 |
| NZ_LXZD00000000 | NZ_VSVO00000000 | NZ_PECX00000000 | NZ_AFMN00000000 |
| NZ_LXZC00000000 | NZ_VSVN00000000 | NZ_SOPE00000000 | NZ_AICL00000000 |
| NZ_NBEY00000000 | NZ_CP017107 | NZ_CP020858 | NZ_AICL00000000 |
| NZ_LXZA00000000 | NZ_VSVL00000000 | NZ_CP020859 | NZ_AICL01000008 |
| NZ_CP024070 | NZ_VSVK00000000 | NZ_CP020860 | NZ_AICL01000008 |
| NZ_NFHF00000000 | NZ_VSVJ00000000 | NZ_NBEL00000000 | NC_006530 |
| NZ_CP024063 | NZ_VSUU00000000 | NZ_NBEK00000000 | NC_007929 |
| NZ_CP024064 | NZ_NBEF00000000 | NZ_LXZM00000000 | NC_007929 |
| NZ_CP024065 | NZ_NBEE00000000 | NZ_VSTJ00000000 | NC_006530 |
| NZ_CP024066 | NZ_VSUR00000000 | NZ_CP007649 | NC_006529 |
| NZ_CP024067 | NZ_VSVC00000000 | NZ_CP007647 | NC_006529 |
| NZ_CP024068 | NZ_VSVB00000000 | NZ_CP007648 | NC_007930 |
| NZ_QAGV00000000 | NZ_VSVA00000000 | NZ_VSTN00000000 | NC_007930 |
| NZ_VSVH00000000 | NZ_VSUZ00000000 | NZ_LXZT00000000 | NZ_CBVR000000000 |
| NZ_VSVG00000000 | NZ_VSUY00000000 | NZ_LXZS00000000 | NZ_CBVR000000000 |
| NZ_VSVF00000000 | NZ_VSUX00000000 | NZ_LXZR00000000 | NZ_CP011403 |
| NZ_VSVW00000000 | NZ_VSUV00000000 | NZ_NBEG00000000 | NZ_CP011405 |
| NZ_VSUS00000000 | NZ_VSUQ00000000 | NZ_VSUT00000000 | NZ CP011404 |

In some embodiments, the present invention provides strains of Lactobacillus casei having the characteristics of a strain selected from the group consisting:

| Strain | Assembly | Strain | Assembly | Strain | Assembly |
|---|---|---|---|---|---|
| ATCC 393 | GCA_000829055.1 | L.cR4 | GCA_011754305.1 | UW4 | GCA_000309745.1 |
| LC5 | GCA_002192215.1 | GCRL 163 | GCA_002091995.1 | A2-362 | GCA_000309625.1 |
| BL23 | GCA_000026485.1 | MJA 12 | GCA_002091975.1 | YNF-5 | GCA_004123005.1 |
| W56 | GCA_000318035.1 | 21/1 | GCA_000309585.1 | BCRC 80156 | GCA_005795975.1 |
| CECT 9104 | GCA_900492555.1 | NBRC 101979 | GCA_007989685.1 | BCRC 17487 | GCA_005795995.1 |
| 12A | GCA_000309565.2 | FAM 20446 | GCA_005864085.1 | DS1_13 | GCA_003052865.1 |
| JCM 1134 | GCA_000615205.1 | A2-362 | GCA_000510825.1 | DS13_13 | GCA_003052855.1 |
| N87 | GCA_001013375.1 | 867_LCAS | GCA_001066695.1 | Z11 | GCA_001885295.1 |
| MGYG-HGUT-02383 | GCA_902386575.1 | AMBR2 | GCA_900185125.1 | B900021 | GCA_001940585.1 |
| BIO5773 | GCA_008868595.1 | DSM 20011 | GCA_001433735.1 | | |

In some embodiments, the present invention provides strains of Bifidobacterium lactis having the characteristics of a strain selected from the group consisting: Bifidobacterium animalis subsp. lactis AD011, Bifidobacterium animalis subsp. lactis ATCC 27536, Bifidobacterium animalis subsp. lactis ATCC 27673, Bifidobacterium animalis subsp. lactis ATCC 27674, Bifidobacterium animalis subsp. lactis B420, Bifidobacterium animalis subsp. lactis BB-12, Bifidobacterium animalis subsp. lactis Bf6, Bifidobacterium animalis subsp. lactis Bi-07, Bifidobacterium animalis subsp. lactis B1-04, Bifidobacterium animalis subsp. lactis B112, Bifidobacterium animalis subsp. lactis BLC1, Bifidobacterium animalis subsp. lactis BS 01, Bifidobacterium animalis subsp. lactis CECT8145, Bifidobacterium animalis subsp. lactis CNCM 1-2494, Bifidobacterium animalis subsp. lactis DSM 10140, Bifidobacterium animalis subsp. lactis HNO19, Bifidobacterium animalis subsp. lactis KLDS2.0603, Bifidobacterium animalis subsp. lactis V9.

In some embodiments, the present invention provides strains of Bifidobacterium longum having the characteristics of a strain selected from the group consisting: Bifidobacterium longum 3_1_37 DFAAB, Bifidobacterium longum AGR2137, Bifidobacterium longum BORI, Bifidobacterium longum D2957, Bifidobacterium longum DJO10A, Bifidobacterium longum E18, Bifidobacterium longum NCC2705, Bifidobacterium longum subsp. infantis including Bifidobacterium infantis VIII-240, Bifidobacterium longum subsp. infantis 157F, Bifidobacterium longum subsp. infantis ATCC 15697=JCM 1222=DSM 20088, Bifidobacterium longum subsp. infantis CCUG 52486, Bifidobacterium longum subsp. infantis EK3, Bifidobacterium longum subsp. Longum, Bifidobacterium longum subsp. longum 1-5B, Bifidobacterium longum subsp. longum 1-6B, Bifidobacterium longum subsp. longum 17-1B, Bifidobacterium longum subsp. longum 2-2B, Bifidobacterium longum subsp. longum 35B, Bifidobacterium longum subsp. longum 44B, Bifidobacterium longum subsp. longum 7-1B, Bifidobacterium longum subsp. longum 72B, Bifidobacterium longum subsp. longum ATCC 55813, Bifidobacterium longum subsp. longum BBMN68, Bifidobacterium longum subsp. longum CECT 7347, Bifidobacterium longum subsp. longum CMCC P0001, Bifidobacterium longum subsp. longum EK13, Bifidobacterium longum subsp. longum EK5, Bifidobacterium longum subsp. longum F8, Bifidobacterium longum subsp. longum GT15, Bifidobacterium longum subsp. longum JCM 1217, Bifidobacterium longum subsp. longum JDM301, Bifidobacterium longum subsp. longum KACC 91563, Bifidobacterium longum subsp. Suillum, Bifidobacterium longum subsp. Suis, Bifidobacterium longum subsp. suis DSM 20211, Bifidobacterium longum X-95

In some embodiments, the present invention provides strains of Bifidobacterium breve having the characteristics of a strain selected from the group consisting: Bifidobacterium breve 12L, Bifidobacterium breve 2L, Bifidobacterium breve 31L, Bifidobacterium breve 689b, Bifidobacterium breve ACS-071-V-Sch8b, Bifidobacterium breve CECT 7263, Bifidobacterium breve DPC 6330, Bifidobacterium breve DSM 20213=JCM 1192, Bifidobacterium breve EX336960VC18, B ifidobacterium breve EX336960VC 19, B ifidobacterium breve EX336960VC21, Bifidobacterium breve EX533959VC21, Bifidobacterium breve HPH0326, Bifidobacterium breve JCM 7017, Bifidobacterium breve JCM 7019, Bifidobacterium breve JCP7499, Bifidobacterium breve MCC 0121, Bifidobacterium breve MCC 0305, Bifidobacterium breve MCC 0476, Bifidobacterium breve MCC 1094, Bifidobacterium breve MCC 1114, Bifidobacterium breve MCC 1128, Bifidobacterium breve MCC 1340, Bifidobacterium breve MCC 1454, Bifidobacterium breve MCC 1604, Bifidobacterium breve MCC 1605, Bifidobacterium breve NCFB 2258, Bifidobacterium breve S27, Bifidobacterium breve UCC2003.

While the bacteria strain used in the present invention is typically a non-genetically modified bacterium, in some examples the bacteria strain is genetically modified to comprise one or more nucleic acid molecule(s) encoding at least one heterologous antigen or a functional fragment thereof.

In some examples the nucleic acid molecule resides extra-chromosomally on, for example, a plasmid vector such as a shuttle vector. Preferably, the plasmid vector would comprise (a) a nucleic acid sequence encoding the heterologous antigen and (b) a control or regulatory sequence operatively linked thereto which is capable of controlling the expression of the nucleic acid when the vector is transformed into a Bacteria strain. In other examples, the nucleic acid molecule inserts into the Bacteria chromosome upon transformation into the Bacteria.

Suitable antigens will be known to the person skilled in the art. Preferably the antigen is an environmental antigen, and may be used either singly or as a combination of two or more such antigens.

The topical composition is useful in preventing and/or treating allergy in a mammal at risk of developing an allergy or having an allergy. In some examples, the allergy is selected from the group consisting of contact dermatitis, chronic inflammatory disorders, allergic atopic disorders, allergic asthma, atopic dermatitis, hyper-IgE syndrome, Omenn's syndrome, psoriasis, hay fever, allergic rhinitis, urticaria, eczema and food allergies.

Accordingly, in a further example the present invention provides a composition for use in preventing or treating allergy in a mammal comprising bacteria, a cell lysate thereof or combination thereof and honey, wherein said bacteria is either killed or inactivated. Optionally, the cell lysate is a whole cell lysate (WCL) of the inactivated bacteria.

In a further example, the present invention provides a composition comprising an Bacteria cell such as an isolated Bacteria cell, or a cell lysate thereof or combination thereof and a pharmaceutically acceptable carrier, wherein said Bacteria cell is inactivated e.g., by virtue of having reduced capacity to colonize the mucosa of a mammal relative to a live Bacteria cell for example having the same genotype as the inactivated cell or by virtue of being incapable of colonizing the mucosa of a mammal. Preferably, the composition is for mucosal delivery. Optionally, the cell lysate is a whole cell lysate (WCL) of the inactivated Bacteria cell.

In another example, the composition comprising inactivated and/or killed Bacteria cells, such as isolated inactivated and/or killed bacteria cells, or a cell lysate thereof, wherein said composition is formulated to be administered topically to a subject for interrupting or slowing or arresting or preventing an atopic march or progression of an atopic march in the subject. For example, the cell lysate is a WCL. In one such example, interrupting or slowing or arresting or preventing an atopic march or progression of an atopic march in the subject comprises delaying or preventing or interrupting or slowing the onset of one or more allergic conditions in the subject.

For example, an allergic condition may comprise allergic eczema, urticaria, hives, rhinitis, wheezing, airway resistance, airway restriction, lung inflammation, food allergy, or asthma. Preferably, an allergic condition comprises airway resistance or airway hyperresponsiveness or hyperreactivity in response to an allergen and wherein the composition is for reducing said airway resistance. Alternatively, or in addition, an allergic condition comprises lung inflammation in response to an allergen and wherein the composition is for reducing said lung inflammation e.g., as characterized by a reduced level of cell infiltrate in lung. Alternatively, or in addition, an allergic condition is characterized by an elevated serum level of allergen-specific IgE antibody and/or an elevated level of one or more inflammatory cytokines in bronchioalveolar lavage (BAL) and/or an elevated level of cell infiltrate in lung. For example, the composition reduces a serum level of allergen-specific IgE antibody and/or a level of one or more inflammatory cytokines in bronchioalveolar lavage (BAL) and/or a level of cell infiltrate in lung relative to a level thereof in a subject exposed to an allergen and not administered said composition. Alternatively, the composition prevents or delays an increase in a serum level of allergen-specific IgE antibody and/or prevents or delays an increase in a level of one or more inflammatory cytokines in bronchioalveolar lavage (BAL) and/or prevents or delays an increase in a level of cell infiltrate in lung in a subject exposed to an allergen.

Heat and Inactivation of Bacteria

Preferably, the composition as described according to any example hereof comprises Bacteria cells or strains which have reduced capability in colonizing the mucosa of a subject relative to live Bacteria cells or strains or are incapable of colonizing the mucosa of a subject. Alternatively, or in addition, the composition according to any example described hereof comprises Bacteria cells or strains which are inactivated e.g., by irradiation such as gamma irradiation and/or ultraviolet irradiation and/or heat treatment and/or chemical means and/or by exposure to acid and/or by exposure to a base and/or by physical means such as pressure and/or by lyophilisation and/or by freeze-thawing. Alternatively, or in addition, the composition according to any example hereof comprises Bacteria cells or strains which are killed e.g., by heat treatment such that the cells are rendered irreversibly metabolically inactive. In another example, the composition according to any example hereof comprises Bacteria cells or strains that have been subjected to a process for inactivating Bacteria cells and a process for killing the Bacteria cells. In one particular example, the inactivated Bacteria cells or strains described according to any example hereof are killed.

Alternatively, or in addition, the composition described according to any example hereof comprises a lysate e.g., WCL of Bacteria cells wherein the cells have been subjected to a process for inactivating Bacteria cells and/or a process for killing the Bacteria cells.

For example, inactivated Bacteria as described according to any example hereof is prepared by exposing live Bacteria cells or strains to irradiation such as gamma irradiation and/or ultraviolet irradiation and/or by exposure to visible light such as wavelengths ranging from about 375 nm to about 500 nm or in a range from about 400 nm to about 420 nm e.g., 405 nm violet light. In one example, inactivated Bacteria as described according to any example hereof is prepared by a process comprising exposing live Bacteria cells or strains to ultraviolet C (UVC) irradiation such as wavelength in a range from about 100 nm to about 280 nm such as about 257.3 nm and/or to ultraviolet B (UVB) irradiation such as wavelength in a range from about 280 nm to about 315 nm and/or to ultraviolet A (UVA) irradiation such as wavelength in a range from about 315 nm to about 400 nm. Preferably, the live Bacteria is exposed to UVC light in a range from about 100 nm to about 280 nm such as about 257.3 nm and/or the live Bacteria is exposed to about 405 nm violet light.

Alternatively, or in addition, inactivated Bacteria as described according to any example hereof is prepared by exposing live Bacteria cells or strains to one or more chemical agents such as formaldehyde and/or β-propiolactone and/or ethyleneimine and/or binary ethyleneimine and/or thimerosal and/or polyethyleneimine functionalized zinc oxide nanoparticles, or derivatives thereof. For example, live Bacteria cells or strains may be inactivated by exposure to formaldehyde at a concentration from about 0.01% to about 1% (w/w) or from about 0.01% to about 0.1% (w/w) or between about 0.025% and about 0.1% (w/w).

Alternatively, or in addition, inactivated Bacteria as described according to any example hereof is prepared by exposing live Bacteria cells or strains to heat treatment such as at temperatures in the range between about 40° C. to about 70° C. or more. Alternatively, or in addition, inactivated Bacteria as described according to any example hereof is prepared by exposing live Bacteria cells or strains to one or more acid(s) or to a low pH environment such as pH 3.0 or lower and/or to one or more base(s) or to high pH environment such as pH 9.0 or higher.

Alternatively, or in addition, inactivated Bacteria as described according to any example hereof is prepared by exposing live Bacteria cells or strains to one or more reducing agent(s) such as sodium bisulfite and/or one or more oxidative agents such as hydrogen peroxide.

Alternatively, or in addition, inactivated Bacteria as described according to any example hereof is prepared by exposing live Bacteria cells or strains to bile salts.

Alternatively, or in addition, inactivated Bacteria as described according to any example hereof is prepared by mutagenesis of live Bacteria cells or strains.

Alternatively, or in addition, inactivated Bacteria as described according to any example hereof is prepared by lyophilizing or freeze-drying live Bacteria cells or strains. Alternatively, or in addition, inactivated Bacteria as described according to any example hereof is prepared by performing one or cycles of freezing and thawing live Bacteria cells or strains.

For example, killed Bacteria as described according to any example hereof is prepared by exposing live and/or inactivated Bacteria cells or strains to heat treatment such as by exposure to temperature of about 60° C. or more for at least about 60 seconds, preferably at a temperature of about 60° C., or about 70° C., or about 80° C., or about 90° C., or about 100° C., or about 110° C., or about 120° C., or about 130° C., or about 140° C., or about 150° C., said temperature exposure being for a period of at least 2 minutes or at least 3 minutes or at least 4 minutes or at least 5 minutes or at least 6 minutes or at least 7 minutes or at least 8 minutes or at least 9 minutes or at least 10 minutes or at least 20 minutes or at least 30 minutes or at least 40 minutes or at least 50 minutes or at least 1 hour or at least 2 hours or at least 3 hours or at least 4 hours or at least 5 hours or at least 6 hours or at least 7 hours or at least 8 hours or at least 9 hours or at least 10 hours or at least 11 hours or at least 12 hours or at least 13 hours or at least 14 hours or at least 15 hours or at least 16 hours or at least 17 hours or at least 18 hours or at least 19 hours or at least 20 hours or at least 21 hours or at least 22 hours or at least 23 hours or at least 1 day or at least 2 days or at least 3 days or at least 5 days or at least 5 days or at least 6 days or at least 7 days. In one preferred example, live and/or inactivated Bacteria is killed by exposure to a single such elevated temperature or by exposure to at least two different elevated temperatures such as by exposure to a first temperature of about 70° C. followed exposure to a second temperature of about 90° C. or about 95° C. In one such preferred example, the live and/or inactivated Bacteria is killed by exposure to temperature of about 70° C. for about 10 minutes followed by exposure to temperature of about 90° C. or about 95° C. for about 5 minutes.

Alternatively, or in addition, killed Bacteria as described according to any example hereof is prepared by exposing live and/or inactivated Bacteria cells or strains to elevated temperatures in the presence of steam and elevated pressure, such as by autoclaving live and/or inactivated Bacteria cells or strains. For example, live and/or inactivated Bacteria is killed by autoclaving the bacterial cells or strains for about 15 minutes at about 121° C. and about 15 psi, or for about 3 minutes at about at 132° C. and about 30 psi.

Alternatively, or in addition, killed Bacteria as described according to any example hereof is prepared by exposing live and/or inactivated Bacteria cells or strains to one or more bactericidal agent(s). For example, live and/or inactivated Bacteria can be subjected to treatment with one or more antibiotics selected from rifampin, amoxicillin, clarithromycin, rifamycin, rifaximin, the rifamycin derivative 3′-hydroxy-5′-(4-isobutyl-1-piperazinyl)benzoxazinorifamycin syn. KRM-1648 and/or the rifamycin derivative 3′-hydroxy-5′-(4-propyl-1-piperazinyl)benzoxazinorifamycin syn. KRM-1657.

Alternatively, or in addition, killed Bacteria as described according to any example hereof is prepared by exposing live and/or inactivated Bacteria cells or strains to irradiation such as gamma irradiation and/or ultraviolet irradiation and/or by exposure to visible light such as wavelengths ranging from about 375 nm to about 500 nm or in a range from about 400 nm to about 420 nm. For example, killed Bacteria is prepared by a process comprising exposing live and/or inactivated Bacteria cells or strains to ultraviolet C (UVC) irradiation such as wavelength in a range from about 100 nm to about 280 nm such as about 257.3 nm and/or to ultraviolet B (UVB) irradiation such as wavelength in a range from about 280 nm to about 315 nm and/or to ultraviolet A (UVA) irradiation such as wavelength in a range from about 315 nm to about 400 nm. Preferably, the live and/or inactivated Bacteria is exposed to UVC light in a range from about 100 nm to about 280 nm such as about 257.3 nm and/or the live and/or inactivated Bacteria is exposed to about 405 nm violet light.

Alternatively, or in addition, killed Bacteria as described according to any example hereof is prepared by sonication e.g., at ultrasonic frequencies such as about 20 kHz or more.

Alternatively, or in addition, killed Bacteria as described according to any example hereof is prepared by mutagenesis of live and/or inactivated Bacteria cells or strains.

Preferably, the killed Bacteria as described according to any example hereof is prepared by first by exposing live Bacteria cells or strains to irradiation such as gamma irradiation and/or ultraviolet irradiation such as UVC light and/or by exposure to visible light such as wavelengths ranging from about 375 nm to about 500 nm or in a range from about 400 nm to about 420 nm, to thereby inactivate Bacteria and then exposing the inactivated Bacteria cells or strains to heat treatment as described according to any example hereof to thereby kill the inactivated Bacteria or render the inactivated Bacteria irreversibly metabolically inactive.

For example, the inactivated Bacteria is exposed to temperature of about 60° C. or more for at least about 60 seconds, preferably at a temperature of about 60° C. or about 70° C. or about 80° C. or about 90° C. or about 100° C. or about 110° C. or about 120° C. or about 130° C. or about 140° C. or about 150° C., said temperature exposure being for a period of at least 2 minutes or at least 3 minutes or at least 4 minutes or at least 5 minutes or at least 6 minutes or at least 7 minutes or at least 8 minutes or at least 9 minutes or at least 10 minutes or at least 20 minutes or at least 30 minutes or at least 40 minutes or at least 50 minutes or at least 1 hour or at least 2 hours or at least 3 hours or at least 4 hours or at least 5 hours or at least 6 hours or at least 7 hours or at least 8 hours or at least 9 hours or at least 10 hours or at least 11 hours or at least 12 hours or at least 13 hours or at least 14 hours or at least 15 hours or at least 16 hours or at least 17 hours or at least 18 hours or at least 19 hours or at least 20 hours or at least 21 hours or at least 22 hours or at least 23 hours or at least 1 day or at least 2 days or at least 3 days or at least 5 days or at least 5 days or at least 6 days or at least 7 days. In one such example, the inactivated Bacteria is exposed to a single such elevated temperature or to at least two different elevated temperatures such as by exposure to a first temperature of about 70° C. e.g., for about 10 minutes, followed by exposure to a second temperature of about 90° C. or about 95° C. e.g., for about 5 minutes.

In one preferred example, the killed Bacteria as described according to any example hereof is prepared by first by exposing live Bacteria cells or strains to ultraviolet irradiation such as UVC light e.g., at about as 257.3 nm to thereby inactivate Bacteria and then exposing the inactivated Bacteria cells or strains to heat treatment as described according to any example hereof to thereby kill the inactivated Bacteria or render the inactivated Bacteria irreversibly metabolically inactive.

Accordingly, in one preferred example, the composition according to any example hereof comprises Bacteria that has been subjected to a process for inactivating Bacteria by irradiation and a process for the killing the inactivated Bacteria by heat treatment.

Alternatively, or in addition, Bacteria as described according to any example hereof is inactivated and/or killed by exposing live or inactivated Bacteria to anaerobic conditions e.g., by changing the atmosphere in which Bacteria is cultured from microaerobic to anaerobic environment for example to mimic the in vivo atmospheric conditions during the washout of Bacteria from the stomach to the lower gut (e.g., small and/or large intestine). For example, live (such as freshly grown) Bacteria is inactivated by exposing (e.g., by growing or incubating) the bacterial cells to anaerobic conditions for about 1 day to about 5 days or more, including for at least about 24 hours, or for at least about 48 hours or at least about 72 hours or at least about 96 hours or at least about 120 hours. In one such example, the live Bacteria cells are inactivated by exposing the cells to anaerobic conditions and by heat treatment of the cells.

In another example, live or inactivated Bacteria as described according to any example hereof is killed by exposing (e.g., by incubation) the live or inactivated bacterial cells to anaerobic conditions for about 1 day to about 5 days or more, including for at least about 24 hours, or for at least about 48 hours or at least about 73 hours or at least about 96 hours or at least about 120 hours.

In one preferred example, the composition according to any example hereof comprises Bacteria that has been subjected to a process for inactivating Bacteria by exposing (e.g., by growing or incubating) the bacterial cells to anaerobic conditions for about 1 day to about 5 days or more, including for at least about 24 hours, or for at least about 48 hours or at least about 73 hours or at least about 96 hours or at least about 120 hours, and a process for the killing the inactivated Bacteria by heat treatment of the cells.

Formulation

In one example, the composition according to any example described hereof is formulated for topical administration to infants, such as to infants who do not have developed lymphoid structures. For example, the topical composition according to any example described hereof is formulated for administration to infants aged between 0 to about 5 years, or between 0 to about 4 years, or between 0 to about 3 years, or between 0 to about 2 years, or between 0 to about 1 year. In one example the composition according to any example described hereof is formulated for topical administration to infants aged between 0 to about 2 years. In another example, the composition is formulated for administration to infants of an age between about 4 months and about 12 months or between about 4 months and about 18 months or about 4 months and about 24 months. In another example, the composition is formulated for topical administration to infants less than about 6 months of age.

In another example, the composition according to any example described hereof is formulated for administration (e.g., by consumption) to children older than about 5 years of age and/or to adolescents and/or to adults.

In another example, the composition according to any example described hereof is formulated for repeated administration, or is administered repeatedly, for example, once per week, or twice per week, or three times per week, or 4 times per week, or 5 times per week, or 6 times per week, or 7 times per week, or more than 7 times per week, or more than twice per day.

In one example, the composition according to any example described hereof is formulated or administered as a multi-dosage unit composition. For example, each dosage of the composition comprises an amount of the Bacteria or a lysate thereof in a range corresponding to between bacteria are present in the topical composition at a concentration of at least about 10⁴, 10⁵, 10⁶, 10⁷, 10⁸ colony forming units (CFU)/gram (g) topical composition or higher, or within a concentration range bounded by any of these values (e.g., 10⁴-10⁸ CFU/g). Where the bacteria are killed bacteria, the concentration of the bacteria added to the composition may be determined prior to killing the bacteria. In other embodiments, the bacteria are present at a concentration of at least about 10⁻⁸, 10⁻⁷, 10⁻⁶, 10⁻⁵, 10⁻⁴ g bacteria/g composition or higher, or within a concentration range bounded by any of these values (e.g., 10⁻⁷-10⁻⁸ g bacteria/g composition). In other embodiments, the bacteria are present at a concentration of at least about 1%, 2%, 3%, 5%, 7%, 10%, 15%, 20% (w/w) or higher or within a range bounded by any of these values (e.g., at a concentration of 3-10% (w/w)).

For example, each dosage of the composition comprises an amount of the Bacteria or a lysate thereof corresponding to at least about 10⁻⁸, 10′, 10⁻⁶, 10⁻⁵, 10⁻⁴ g bacteria/g composition or higher, or within a concentration range bounded by any of these values (e.g., 10⁻⁷-10⁻⁸ g bacteria/g composition). In one example, the composition according to any example described hereof is formulated for administration daily, or is administered daily, wherein a daily dosage of said composition comprises an amount of the Bacteria or a lysate thereof in a range corresponding to at least about 10⁴, 10⁵, 10⁶, 10⁷, 10⁸ colony forming units (CFU)/gram (g) topical composition or higher. For example, each daily dosage of the composition comprises an amount of the Bacteria or a lysate thereof corresponding to a concentration of at least about 10⁻⁸, 10⁻⁷, 10⁻⁶, 10⁻⁵, 10⁻⁴ g bacteria/g composition or higher.

In one example, the composition according to any example described hereof is formulated for administration, or is administered, over a period of at least about 2 weeks or at least about 4 weeks or at least about 6 weeks or at least about 8 weeks or at least about 10 weeks or at least about 11 weeks or at least about 12 weeks or at least about 13 weeks at least about 14 weeks or at least about 15 weeks or at least about 16 weeks or at least about 17 weeks or at least about 18 weeks or at least about 19 weeks or at least about 20 weeks or at least about 21 weeks or at least about 22 weeks or at least about 23 weeks or at least about 24 weeks or at least about 25 weeks, or at least about 6 months, or at least about one year or more than one year. Preferably, the composition according to any example described hereof is formulated for administration, or is administered, over a period of at least about 13 weeks or at least about 3 months.

In one example, the composition according to any example described hereof is formulated for administration, or is administered, in absence of honey and/or wherein said composition does not comprise honey.

Dosaging

In another example, the composition or a dosage (e.g., daily dosage) of the composition according to any example described hereof promotes a balanced development of an immune system in a juvenile subject. In another example, the composition or a dosage (e.g., daily dosage) of the composition according to any example described hereof promotes acquisition of adaptive immunity and/or innate immunity in a subject. In another example, the composition or a dosage (e.g., daily dosage) of the composition according to any example described hereof promotes or enhances CD1d receptor activation and/or CD4-negative and CD8-negative natural killer (NK) cells in a subject. In another example, the composition or a dosage (e.g., daily dosage) of the composition according to any example described hereof promotes or enhances γδ T-cell activation. In another example, the composition or a dosage (e.g. daily dosage) of the composition according to any example described hereof promotes or enhances mucosal immunity involving immune recognition and presentation to antigen-presenting cells (APCs). In another example, the composition or a dosage (e.g., daily dosage) of the composition according to any example described hereof promotes a balanced Th1/Th2 immune response to one or more allergens.

In another example, the composition according to any example described hereof comprises an amount of killed Bacteria cells and/or inactivated Bacteria cells and/or a cell lysate of said killed or inactivated cells.

The present invention clearly extends to the manufacture of a composition for use in preventing or treating allergy in a mammal, said manufacture comprising use of an isolated Bacteria cell, a cell lysate thereof, wherein said Bacteria cell is either killed or incapable of colonizing the skin of said mammal.

In one example, the present invention relates to use of an Bacteria cell such as an isolated Bacteria cell, and/or a cell lysate thereof or a combination thereof, wherein said Bacteria cell is inactivated or killed in the preparation of a composition for preventing or treating allergy in a mammal e.g., wherein the inactivated Bacteria cell does not have the same capacity of a live Bacteria cell having the same genotype to colonize the mucosa of a mammal to which it is administered or wherein the inactivated or killed Bacteria is incapable of colonizing the mucosa of a mammal to which it is administered. Optionally, wherein the cell lysate is a whole cell lysate (WCL) of the inactivated or killed Bacteria cell.

In another example, the present invention relates to use of an inactivated and/or killed Bacteria, such as an isolated and inactivated and/or killed Bacteria, or a cell lysate thereof or a combination thereof in the preparation of a composition according to any example described hereof for interrupting or slowing or arresting or preventing an atopic march or progression of an atopic march in the subject. Optionally, wherein the cell lysate is a whole cell lysate (WCL) of the inactivated and/or Bacteria.

In another example, the present invention relates to use of an inactivated and/or killed Bacteria, such as an isolated and inactivated and/or killed Bacteria, or a cell lysate thereof or a combination thereof in the preparation of a composition according to any example described hereof for delaying or preventing or interrupting or slowing the onset of one or more allergic conditions in the subject. Optionally, wherein the cell lysate is a whole cell lysate (WCL) of the inactivated and/or killed Bacteria.

In some embodiments, the mammal is a naive mammal. Thus, in a further example, the present invention provides a method of preventing allergy in an immunologically naive mammal at risk of developing said allergy, said method comprising the step of; (i) identifying a mammal at risk of developing an allergy, (ii) administering to said mammal a composition comprising an isolated Bacteria cell, a cell lysate thereof or combination thereof and a pharmaceutically accepted carrier, wherein said Bacteria cell is either killed or incapable of colonizing the mucosa of said mammal and (iii) allowing sufficient time to elapse to enable anergy to develop.

Before

In another example of the method according of the present invention according to any example described hereof the administration or the Bacteria or the lysate or composition promotes development of a balanced development of an immune system in a juvenile subject.

In another example of the method according to any example described hereof the administration or the Bacteria or the lysate or composition promotes acquisition of adaptive immunity in a subject.

In another example of the method according to any described hereof, the administration or the Bacteria or the lysate or composition promotes acquisition of adaptive immunity in a subject.

In another example of the method according to any described hereof, the administration or the Bacteria or the lysate or composition promotes or enhances CD1d receptor activation and/or CD4-negative and CD8-negative natural killer (NK) cells.

In another example of the method according to any described hereof, the administration or the Bacteria or the lysate or composition promotes or enhances γδ T-cell activation.

In another example of the method according of the present invention according to any described hereof, the administration or the Bacteria or the lysate or composition promotes or enhances mucosal immunity involving immune recognition and presentation to antigen-presenting cells (APCs).

In another example of the method according to any example hereof, the administration or the Bacteria or the lysate or composition promotes a balanced Th1/Th2 immune response to one or more allergens.

In another example of the method according to any described hereof, the subject is asymptomatic for eczema, or asymptomatic for allergy, or asymptomatic for asthma, and wherein said method prevents a subsequent onset of eczema and/or allergy and/or asthma in the subject e.g., following exposure of the subject to an allergen. In one such example, the method comprises administering an isolated and inactivated Bacteria to a juvenile subject to prevent eczema in the infant or a subsequent onset of allergy or asthma in later life. Alternatively, the method comprises administering the isolated and inactivated Bacteria to an adolescent or adult subject to prevent eczema in the subject or a subsequent onset of allergy or asthma in later life in the subject. However, a subsequent onset of eczema and/or allergy and/or asthma may be induced in an untreated subject to whom the Bacteria or the composition has not been administered by exposure of the untreated subject to an allergen. For example, the allergen is an environmental allergen, pollen allergen, dust mite allergen, animal allergen, or chemical allergen.

In another example of the method according to any described hereof, the subject has suffered previously from one or more incidences of allergic eczema, allergy, or asthma, and wherein said method prevents a subsequent attack or reduces severity of a subsequent attack in the subject. In one such example, the method comprises administering the inactivated and/or killed Bacteria, such as isolated inactivated and/or killed Bacteria, or a cell lysate thereof or a combination thereof, to an adolescent or adult subject that has suffered previously from allergic eczema and/or allergy and/or asthma, to thereby prevent a subsequent attack or reduce severity of a subsequent attack, optionally to prevent or slow further atopic march in the subject. Alternatively, the method comprises administering the inactivated and/or killed Bacteria such as isolated inactivated and/or killed Bacteria, or a cell lysate thereof or a combination thereof, to an adolescent or adult subject that has suffered previously from allergic eczema and/or allergy and/or asthma, to thereby prevent a subsequent attack or reduce severity of a subsequent attack, optionally to prevent or slow further atopic march in the subject. However, a subsequent attack of eczema and/or allergy and/or asthma may be inducible in an untreated subject to whom the Bacteria or the composition has not been administered by exposure of the untreated subject to an allergen. For example, the allergen is an environmental allergen, pollen allergen, dust mite allergen, animal allergen, or chemical allergen.

In another example of the method according to any described hereof, administration of an inactivated and/or killed Bacteria, such as isolated inactivated and/or killed Bacteria, or a cell lysate thereof or a combination thereof, to a subject reduces the incidence of allergic immune responses in a population of subjects.

In another example of the method according to any described hereof, administration of an inactivated and/or killed Bacteria, such as isolated inactivated and/or killed Bacteria, or a cell lysate thereof or a combination thereof, to a subject reduces the incidence of allergic immune responses in adolescent and/or adult members of the population treated when they were juveniles.

In a further example, the present invention provides a method of treating allergy in a mammal comprising the step of administering to said mammal an effective amount of a composition comprising an isolated Bacteria cell, a cell lysate thereof or combination thereof and a pharmaceutically accepted carrier, wherein said Bacteria cell is either killed or incapable of colonizing the mucosa of said mammal, wherein said composition, upon administration, provides protective immunity against said allergy. The mammal or subject includes a dog, a cat, a livestock animal, a primate or a horse.

In some embodiment, the mammal or subject is a human subject. Preferably, the human subject is below the age of about 5. More preferably, the human subject is below the age of 2 years.

In one example, the present invention provides a kit for treating and/or preventing allergy in a mammal comprising (i) a composition according to any example hereof and (ii) instructions for use in a method according to any one of examples hereof.

The present invention also clearly extends to a kit for treating and/or preventing allergy in a subject, said kit comprising (i) the inactivated and/or killed Bacteria or the lysate or the composition as described according to any example hereof, and optionally (ii) instructions for use in a method or use according to any one of examples hereof. For example, the kit is for use in preventing or attenuating allergic airway hyper-responsiveness in lungs of a subject following exposure of the subject, such as an asthmatic subject, to an allergen. Alternatively, or in addition, the kit is for use in preventing or alleviating airway resistance or airway hyper-responsiveness in lungs of an asthmatic subject following exposure of said subject to an allergen. Alternatively, or in addition, the kit is for use in preventing an allergic immune response to an allergen in a subject or reducing severity or incidence of an allergic immune response to an allergen in a subject. Alternatively, or in addition, the kit is for use in interrupting or slowing or arresting or preventing an atopic march or progression of an atopic march in a subject. Alternatively, or in addition, the kit is for use in delaying or preventing or interrupting or slowing the onset of one or more allergic conditions in a subject, for example wherein the one or more condition(s) is/are characterized by an elevated serum level of allergen-specific IgE antibody and/or an elevated level of one or more inflammatory cytokines in bronchioalveolar lavage (BAL) and/or an elevated level of cell infiltrate in lung of the subject. Alternatively, or in addition, the kit is for use in delaying or preventing or interrupting or slowing the onset of one or more of allergic eczema, urticaria, hives, rhinitis, wheezing, airway resistance, airway restriction, lung inflammation, food allergy, or asthma in a subject. Alternatively, or in addition, the kit is for delaying or preventing or interrupting or slowing the onset of airway resistance and/or lung inflammation response to an allergen in a subject. Alternatively, or in addition, the kit is for delaying or preventing or interrupting or slowing cell infiltration into lung of a subject in response to an allergen.

List of Probiotic Organisms and Commercial Sources

Bacillus coagulans Ganeden Inc., USA ATCC 25527 Bacteroides adolescentis CNCM I-2168 Bifidobacterium animalis ATCC (American Tissue Type Collection, Manassas, Va.) B. bifidum ATCC 15700 B. breve ATCC DPTC 001 B. breve R-070 Institut Rosell Inc., Montreal, Quebec, Canada ATCC 15697 B. infantis ATCC DPTC 047 B. infantis BBI Chr. Hansen, Milwaukee, Wis. DPTC 002 B. lactis Bb12 (ATCC27536) Chr. Hansen B. lactis NCC2818 (CNCMI-3446) ATCC 15708 B. longum ATCC DPTC 004 B. longum BB46 Chr. Hansen DPTC 003 B. longum BBL Chr. Hansen B. longum NCC490 (CNCM 1-2170) (a.k.a., B. longum Bb536 or Morinaga strain) DPTC 036 B. spp. Rolly fermented milk, Snow Brand Escherischia coli M-17 BioBalance Inc., USA E. coli K12 E. coli Nissle ATCC 4356 Lactobacillus acidophilus ATCC ATCC 700396 L. acidophilus ATCC DPTC 025 L. acidophilus Mil Mil fermented milk, Yakult, Tokyo, Japan DPTC 049 L. acidophilus Mil Mil fermented milk, Yakult DPTC 046 L. acidophilus AS-1 Quest International, Rochester, Minn. DPTC 027 L. acidophilus DDS-1 Capsule supp., Natren Inc., Westlake Village, CA DPTC 010 L. acidophilus HP10 Northeast Nutraceuticals, S. Boston, Mass. DPTC 011 L. acidophilus HP100 Northeast Nutraceuticals DPTC 012 L. acidophilus HP101 Northeast Nutraceuticals DPTC 013 L. acidophilus HP102 Northeast Nutraceuticals DPTC 014 L. acidophilus HP103 Northeast Nutraceuticals DPTC 015 L. acidophilus HP104 Northeast Nutraceuticals DPTC 048 L. acidophilus HP15 Northeast Nutraceuticals DPTC 005 L. acidophilus NCFM Rhodia Inc., Madison, Wis. DPTC 006 L. acidophilus NCFM N.C. State University, Raleigh, N.C. DPTC 007 L. acidophilus PIM703 Chr. Hansen DPTC 008 L. acidophilus SBT2062 Snow Yogurt+2, Snow Brand L. alimentarius ATCC 33620 L. amylovorus ATCC ATCC 393 L. casei ATCC DPTC 051 L. casel DN-114 001 Actimel Original fermented milk, Danone, Paris, France DPTC 034 L. casei LC10 Rhodia DPTC 035 L. casei PIM661 Chr. Hansen DPTC 033 L. casel Shirota Joie fermented milk drink, Yakult DPTC 030 L. casei Shirota Health drink produced by Yakult ATCC 33820 L. crispatus ATCC DPTC 009 L. crispatus BG2FO4 NCSU L. curvatus ATCC 11842 L. delbrueckii ssp. bulgaricus ATCC DPTC 020 L. delbrueckii ssp. bulgaricus 2038 Yogurt, Meiji Milk Products Co. Ltd., Tokyo, Japan DPTC 021 L. delbrueckii ssp. bulgaricus 2038 Yogurt, Meiji DPTC 019 L. delbrueckii ssp. bulgaricus MR120 Rhodia DPTC 022 L. delbrueckii ssp. bulgaricus PIM695 Chr. Hansen L. delbrueckii ssp. lactis DPTC 045 L. rhamnosus MX1 University of Western Ontario, London, Ontario, Canada ATCC 33199 L. gallinarum ATCC ATCC 33233 L. gasseri ATCC DPTC 026 L. gasseri ADH NCSU DPTC 016 L. helveticus MR220 Rhodia DPTC 017 L. helveticus NCK388 NCSU ATCC 33200 L. johnsonii ATCC DPTC 028 L. johnsonii 11088 (NCK 088) NCSU DPTC 029 L. johnsonii La-1 Nestle´, Lausanne, Switzerland L. johnsonii CNCM 1-1225 DPTC 018 L. lactis San Chr. Hansen ATCC 25302 L. paracasei ATCC L. paracasei Lpc-37 L. paracasei ST11 NCC 2461 (a.k.a., CNCM 1-2116) ATCC 23272 L. reuteri ATCC DPTC 037 L. reuteri 1063-S Biogaia Biologics, Stockholm, Sweden DPTC 038 L. reuteri 11284 Biogaia Biologics DPTC 039 L. reuteri SD2112 Biogaia Biologics DPTC 040 L. reuteri T-1 Biogaia Biologics ATCC 7469 L. rhamnosus ATCC DPTC 042 L. rhamnosus GR-1 University of Western Ontario DPTC 043 L. rhamnosus R-011 Institut Rosell DPTC 044 L. rhamnosus R-049 Institut Rosell ATCC 53103 L. rhamnosus GG ATCC Lactococcus lactis Leuconostoc mesenteroides, subspecies cremoris Proprionibacterium freudenrichii, subspecies shermanii JS ATCC 10556 Streptococcus salivarius S. mitis S. oralis S. sanguis S. thermophiles S244 ATCC Staphylcoccus carnosus S. xylosus Vitreoscilla fihforrnis Yeast Lyophilized yeast extract Centro Ricerche YOMO, Milan Saccharomyces cerevisiae Health Sciences USA ATCC 74012 S. boulardii Biocodex, Gentilly, France; ATCC ATCC MYA-797 S. boulardii ATCC S. subtilis.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Testing for Reduction of Hyper-Responsiveness

The method for the general reduction in hyper-responsiveness of an individual to one or more allergens thereby delaying or preventing or interrupting or slowing the onset of one or more allergic conditions. The reduced hypersensitivity may be demonstrated by reduced sensitivity of a subject to a specific allergen e.g., an accepted model allergen of hypersensitivity e.g., ovalbumin and/or ragweed administered as a challenge to murine animals e.g., BALB/c or C57/BL/6 or SJL/J mice, in an aerosolized form or by gavage. See e.g., Renz et al., J. Allergy Clin. Immunol. 89:1127-1138 (1992); Renz et al., J. Immunol. 151:1907-1917 (1993); Saloga et al., J. Clin. Invest. 91:133-140 (1993); Larsen et al., J. Clin. Invest. 89:747-752 (1992); Oshiba et al., J. Clin, Invest. 97: 1938-1408 (1996).

Testing for Infants susceptible to AM

Sample Collection, Protein Extraction and Mass Spectrometry

Patients were directed to avoid showering on the day of the collection. A total of 20 consecutive D-SQUAME® tape strips (22 mm diameter, CuDerm, Dallas, Tex., USA) were performed on the volar surface of right forearm at the age of 2 months. On application of the first tape disc, 4 marks were placed around the disc with a pen so that subsequent discs could be applied to the same location. Each tape disc was placed adhesive side up in its own 6-well plate and then frozen at −80° C. Proteins were extracted by using a buffer composed of 0.01% 34341,1-bisalkyloxyethyl] pyridin-1-yl)propane-1-sulfonate (Protein Discovery, Knoxville, Tenn., USA) in 50 mmol/L ammonium bicarbonate with 1× HALT protease inhibitors, EDTA-free (Thermo Fisher Scientific, Rockford, Ill., USA), and 50 mmol/L dithiothreitol (Bio-Rad, Hercules, Calif., USA) and incubated on a rocker at room temperature for 1 hour. Extraction buffer was pooled from tape disc, transferred into polypropylene 1.5 mL microcentrifuge tubes, lyophilized, and stored at −80° C.

Proteins were precipitated in 300 mL ice-cold precipitation buffer consisting of 0.1% formic acid in 80:20 methanol:water (VWR, West Chester, Pa., USA; and Thermo Fisher Scientific, respectively). Samples were incubated at −20° C. and vortexed for 30 seconds every 10 minutes for 1 hour, then centrifuged at 18,000 g at 4° C. for 20 minutes. The supernatant was removed, and the protein pellet was resuspended in 8 mol/L urea (Sigma Ultra, St Louis, Mo., USA) in 100 mmol/LTRIS HCl (pH 8.5; Thermo Fisher Scientific). Proteins were reduced with 5 mmol/L TCEP (tris[2-carboxyethyl]phosphine) (Thermo Fisher Scientific) for 20 minutes and alkylated with 500 mmol/L iodoacetamide (Bio-Rad) for 15 minutes. Urea was diluted to 2 mol/L with 100 mmol/L TRIS HCl (pH 8.5), and samples were incubated at 37° C. overnight with 100 ng trypsin (Trypsin Gold; Promega, Madison, Wis., USA). Samples were then cleaned by using PepClean C-18 spin columns (Thermo Fisher Scientific) following the manufacturer's protocol. All reagents were of the highest grade available for mass spectrometry.

Mass spectrometry was carried out as previously described by Broccardo C J, et al. (J Allergy Clin Immunol 2009; 124:1113-5 el-11). Samples were run in triplicate on an Agilent 1200 series HPLC (Agilent Technologies, Santa Clara, Calif., USA) and Agilent ETD ion trap (model 6340) mass spectrometer with an HPLC chip to evaluate the expression of filaggrin, alpha enolase, corneodesmosin, fatty acid binding protein, serpin B3, transglutaminase 3, and thymic stromal lymphopoietin (TSLP).

Randomized Controlled Trial to Prevent Atopic Dermatitis, Food Allergy and Sensitization in Infants with a Family History of Allergic Disease.

Infants (n=760) with a family history of allergic disease will be recruited from hospitals, clinics, and research centers. The primary outcomes are as follows: the presence of AD, assessed using the UK Working Party criteria, and food allergy using food challenge, in the first 12 months of life as assessed by a blinded study outcome assessor. Secondary outcomes are as follows: food sensitization (skin prick test), skin barrier function, AD severity, the presence of new onset AD after treatment cessation (between 6 and 12 months) and the presence of parent reported AD/eczema. Parents or guardians will provide written informed consent.

### REFERENCES

1. Lack G. Epidemiologic risks for food allergy. Journal of Allergy and Clinical Immunology. 2008;121(6): 1331- 1336.
2. Boralevi F, Hubiche T, Leaute-Labreze C et al. Epicutaneous aeroallergen sensitization in atopic dermatitis infants—determining the role of epidermal barrier impairment. Allergy. 2008;63(2):205-210.
3 Brown S J & McLean W H. One remarkable molecule: filaggrin. J Invest Dermatol. 2012; 132(3 Pt 2):751-762. PMCID: PMC3378480.
4 Irvine A D, McLean W H I & Leung D Y M. Filaggrin Mutations Associated with Skin and Allergic Diseases. N. Engl. J. Med. 2011; 365(14):1315-1327.
5 Tan H T T, Ellis J A, Koplin J J et al. Filaggrin loss-of-function mutations do not predict food allergy over and above the risk of food sensitization among infants. J. Allergy Clin. Immunol. 2012; 130(5):1211-1213.
6 Thyssen J P & Kezic S. Causes of epidermal filaggrin reduction and their role in the pathogenesis of atopic dermatitis. J Allergy Clin Immunol. 2014; 134(4):792-799.
7 Spergel J M, Mizoguchi E, Brewer J P, Martin T R, Bhan A K & Geha R S. Epicutaneous sensitization with protein antigen induces localized allergic dermatitis and hyperresponsiveness to methacholine after single exposure to aerosolized antigen in mice. J Clin Invest. 1998; 101(8):1614-1622. PMCID: PMC508742.
8 Chan S M, Turcanu V, Stephens A C, Fox A T, Grieve A P & Lack G. Cutaneous lymphocyte antigen and alpha4beta7 T-lymphocyte responses are associated with peanut allergy and tolerance in children. Allergy. 2012; 67(3):336-342.
9 Weissler K A, Rasooly M, DiMaggio T et al. Identification and analysis of peanut-specific effector T and regulatory T cells in children allergic and tolerant to peanut. J Allergy Clin Immunol. 2018; 141(5):1699-1710 e1697. PMCID: PMC5938104.
10 Brough H A, Santos A F, Makinson K et al. Peanut protein in household dust is related to household peanut consumption and is biologically active. J Allergy Clin Immunol. 2013; 132(3):630-638.
11 Brough H A, Liu A H, Sicherer S et al. Atopic dermatitis increases the effect of exposure to peanut antigen in dust on peanut sensitization and likely peanut allergy. J Allergy Clin Immunol. 2015; 135(1):164-170. PMCID: PMC4282723.
12 du Toit G, Sayre P H, Roberts G et al. Allergen specificity of early peanut consumption and effect on development of allergic disease in the Learning Early About Peanut Allergy study cohort. J Allergy Clin Immunol. 2018; 141(4):1343-1353. PMCID: PMC5889963.
13 Perkin M R, Logan K, Tseng A et al. Randomized Trial of Introduction of Allergenic Foods in Breast-Fed Infants. N Engl J Med. 2016; 374(18):1733-1743.
14 Bellach J, Schwarz V, Ahrens B et al. Randomized placebo-controlled trial of hen's egg consumption for primary prevention in infants. J Allergy Clin Immunol. 2017; 139(5):1591-1599 e1592.
15 Berin M C & Sampson H A. Mucosal immunology of food allergy. Curr Biol. 2013;23(9):R389-400. PMCID: 3667506.
16 Williams M R & Gallo R L. The Role of the Skin Microbiome in Atopic Dermatitis. Curr Allergy Asthma Rep. 2015;15(11):65.
17 Totte J E, van der Feltz W T, Hennekam M, van Belkum A, van Zuuren E J & Pasmans S G. Prevalence and odds of Staphylococcus aureus carriage in atopic dermatitis: a systematic review and meta-analysis. Br J Dermatol. 2016; 175(4):687-695.
18 Syed A, Garcia M A, Lyu S C et al. Peanut oral immunotherapy results in increased antigen-induced regulatory T-cell function and hypomethylation of forkhead box protein 3 (FOXP3). J Allergy Clin Immunol. 2014; 133(2):500-510. PMCID: PMC4121175.
19 Schneider L, Tilles S, Lio P et al. Atopic dermatitis: a practice parameter update 2012. J Allergy Clin Immunol. 2013; 131(2):295-299.e291-227.
20 Turcanu V, Brough H A, Du Toit G et al. Immune mechanisms of food allergy and its prevention by early intervention. Curr Opin Immunol. 2017; 48:92-98. PMCID: PMC5682223.
21 Abdel-Gadir A, Schneider L, Casini A et al. Oral immunotherapy with omalizumab reverses the Th2 cell-like programme of regulatory T cells and restores their function. Clin Exp Allergy. 2018; 48(7):825-836. PMCID: PMC6021220.
22 Yu W, Freeland D M H & Nadeau KC. Food allergy:immune mechanisms, diagnosis and immunotherapy. Nat Rev Immunol. 2016; 16(12):751-765. PMCID: PMC5123910.
23 Ebbo M, Crinier A, Vely F & Vivier E. Innate lymphoid cells: major players in inflammatory diseases. Nat Rev Immunol. 2017; 17(11):665-678.

All publications and patent applications mentioned in this specification are herein incorporated by reference to the same extent as if each individual publication or patent application was specifically and individually indicated to be incorporated by reference.

What is claimed is:

1. A method of interrupting or slowing or arresting or preventing an atopic march or progression of an atopic march in a human subject having or susceptible to atopic march, the method comprising, administering to the subject an effective amount of a composition comprising an inactivated or killed bacteria or a cell lysate thereof and honey; wherein the Bacteria are Lactobacillus Acidophilus and Lactobacillus Plantarum.

2. The method of claim 1, wherein said method comprises administering a daily dosage of the bacteria or cell lysate wherein each dose comprises an amount of bacteria in a range corresponding to 1 million colony forming units per gram in a topical composition.

3. The method of claim 2, wherein the bacteria or cell lysate thereof is administered to a juvenile human subject to prevent eczema in the juvenile or a subsequent onset of allergy or asthma in later life.

4. The method of claim 3, wherein the bacteria or cell lysate thereof is administered over a period of at least about 2 weeks.

5. The method of claim 3, wherein the bacteria or cell lysate thereof is administered over a period of at least about 4 weeks.

6. The method of claim 3, wherein the bacteria cells or cell lysate thereof is administered over a period of at least about 6 weeks.

7. The method of claim 6, wherein said method prevents or delays an increase in a serum level of allergen-specific IgE antibody and/or prevents or delays an increase in a level of one or more inflammatory cytokines in bronchioalveolar lavage (BAL) and/or prevents or delays an increase in a level of cell infiltrate in lung in a human subject exposed to an allergen.

8. The method of claim 7, wherein the composition further comprises turmeric.

9. The method of claim 8, wherein the composition further comprises vitamin B12.

10. A method for preventing an allergic immune response to an allergen or reducing severity or incidence of an allergic immune response to an allergen in a human subject having or susceptible to an allergic immune response to an allergen comprising administering to the subject a therapeutically effective amount of killed or inactivated bacteria or a cell lysate thereof and honey; wherein the bacteria is selected from the group consisting of Lactobacillus, Bifzdobacterium, or Streptococcus.

11. The method of claim 10, wherein said method comprises administering a daily dosage of the bacteria or cell lysate wherein each dose comprises an amount of bacteria in a range corresponding to 1 million colony forming units per gram in a topical composition.

12. The method of claim 11, wherein the bacteria or cell lysate thereof is administered to a juvenile human subject to prevent eczema in the juvenile or a subsequent onset of allergy or asthma in later life.

13. The method of claim 12, wherein the bacteria or cell lysate thereof is administered over a period of at least about 2 weeks.

14. The method of claim 12, wherein the bacteria or cell lysate thereof is administered over a period of at least about 4 weeks.

15. The method of claim 12, wherein the bacteria cells or cell lysate thereof is administered over a period of at least about 6 weeks.

16. The method of claim 15, wherein said method prevents or delays an increase in a serum level of allergen-specific IgE antibody and/or prevents or delays an increase in a level of one or more inflammatory cytokines in bronchioalveolar lavage (BAL) and/or prevents or delays an increase in a level of cell infiltrate in lung in a human subject exposed to an allergen.

17. The method of claim 16, wherein the composition further comprises turmeric.

18. The method of claim 17, wherein the composition further comprises vitamin B12.
